(19) United States
(12) Patent Application Publication
(10) Pub. No.: US 2018/0250404 A1
(43) Pub. Date: Sep. 6, 2018

(54) HIGH-Z NANOPARTICLES AND THE USE THEREOF IN RADIATION THERAPY

(71) Applicant: Board of Regents, The University of Texas System, Austin, TX (US)

(72) Inventors: Sunil KRISHNAN, Houston, TX (US); Pankaj SINGH, Houston, TX (US)

(73) Assignee: Board of Regents, The University of Texas System, Austin, TX (US)

(21) Appl. No.: 15/758,437

(22) PCT Filed: Sep. 15, 2016
(86) PCT No.: PCT/US2016/051926

§ 371 Date: Mar. 8, 2018

Related U.S. Application Data
(60) Provisional application No. 62/220,379, filed on Sep. 18, 2015.

(51) Int. Cl. A61K 41/00; A61K 33/24; A61K 47/68; C07K 16/28; C07K 16/30; A61P 35/00; A61K 47/60; A61K 45/06; A61K 47/69; A61N 5/10

(52) U.S. Cl. CPC A61K 41/0038; A61N 2005/1098; A61K 47/6849; A61K 47/6851; C07K 16/2863; C07K 16/30; A61P 35/00; A61K 47/60; A61K 45/06; A61K 47/6929; A61N 5/10; C07K 2317/24; C07K 2317/77; A61N 2005/1087; A61K 33/24

(57) ABSTRACT

Methods for sensitizing target cells to ionizing radiation are provided comprising the administration of high-Z particles (e.g., gold nanoparticles) in conjunction with a de-aggregation agent. In some aspects, particles comprise a targeting molecule to enable cellular uptake by the target cells. Pharmaceutical compositions comprising high-Z particles and de-aggregation agents are also provided.

This application claims the benefit of U.S. Provisional Patent Application No. 62/220,379, filed Sep. 18, 2015, the entirety of which is incorporated herein by reference.

The invention was made with government support under Grant No. 5R01CA155446 awarded by the National Institute of Health. The government has certain rights in the invention.

### BACKGROUND OF THE INVENTION

### 1. Field of the Invention

The present invention relates generally to the field of biology and medicine. More particularly, it concerns cancer therapy.

### 2. Description of Related Art

Radiation therapy is a long-established and effective component of modern cancer therapy for localized disease. However, the ultimate utility of radiation therapy is limited by the fact that some cancer cells are resistant to ionizing radiation. Additionally, the delivery of the ionizing radiation through healthy tissue or beyond the tumor margin limits the radiation dose and may result in unwanted side effects.

In recent years, intravenously administered nanoparticles (NPs) have shown great promise as anti-cancer agents. These NPs accumulate preferentially within tumors largely as a result of their size and passive extravasation from the leaky, chaotic and immature vasculature of tumors—a phenomenon referred to as the “enhanced permeability and retention” (EPR) effect (Dvorak, et al, 1988; Unezaki, et al. 1996; Maeda, et al. 2003). Such nanoparticles may serve individually as therapeutic agents or serve as carriers for drugs or toxins to effect therapy. The functional possibilities of nanoparticles have offered great promise to the field of medicine but have so far failed to produce clinically translatable results. One of their potential uses has been radiation dose enhancement by particles made of high atomic number (Z) elements such as gold. Several studies have demonstrated radiation dose enhancement in the presence of gold nanoparticles (GNP) resulting in substantial tumor regression and long term survival in tumor-bearing mice (Hainfeld, et al, 2004; Hainfeld, et al, 2008; Doh, et al, 2013) generating great excitement in the field of oncology. Unfortunately, enthusiasm for clinical translation of this strategy is dampened by (i) the high intratumoral GNP concentrations (˜1 mg/g tissue) needed, (ii) the strong dependence on the photon beam energy (kilovoltage (kV) x-rays), as predicted by Monte Carlo (MC) simulations (Cho, et al, 2005; Jones, et al, 2010), to achieve a significant (˜>10%) dose enhancement at a macroscopic scale, (iii) the requirement of almost simultaneous administration of GNPs and radiation, and (iv) the lack of an understanding of underlying biological mechanisms driving the radiosensitization. Thus, there is a need for new, more effective, nanoparticle-radiosensitization methods.

### SUMMARY OF THE INVENTION

Embodiments of the present invention provide nanoparticles as radiosensitizing agents to enhance the effectiveness of radiation therapy. In a first embodiment there is provided a method sensitizing target cells in a subject to a radiation therapy comprising: administering to the subject an effective amount of high-Z particles, the high-Z particles comprising a targeting molecule that binds to target cells and a high-Z element; and administering to the subject an effective amount of a de-aggregation agent that reduces intracellular aggregation (or increases intracellular dispersion) of said high-Z particles. In a further embodiment there is provided a method sensitizing a tumor in a subject to a radiation therapy comprising: administering to the subject an effective amount of high-Z particles, the high-Z particles comprising a high-Z element and, optionally, a targeting molecule that binds to tumor cells; and administering to the subject an effective amount of a de-aggregation agent that reduces intracellular aggregation (or increases intracellular dispersion) of said high-Z particles. In some aspects, the high-Z particles are administered before, after or essentially simultaneously with the de-aggregation agent. For example, the high-Z particles and the de-aggregation agent can be comprised in the same composition. In further aspects, the high-Z particles contain or are conjugated to a de-aggregation agent.

Aspects of the invention provide methods for radiosensitization. In certain aspects, a method of the embodiments further comprises irradiating the target cells (or tumors) with ionizing radiation. For example a method can comprise administering an effective amount of high-Z particles, the high-Z particles comprising a targeting molecule that binds to target cells and a high-Z element; administering an effective amount of a de-aggregating agent and irradiating the target cells with ionizing radiation. In certain aspects, the targeting molecule results in the internalization of the high-Z particles upon binding with the target cells.

In some aspects, a high-Z element of the embodiments is gold, silver, iodine, gallium, barium, iron, gadolinium, platinum, hafnium, bismuth or combinations thereof. In certain aspects, the high-Z particles comprise nanorods, nanoshells, colloids, nanocages, nanotriangles, or nanoprisms. In further aspects, the high-Z particles have an average diameter of between about 1 nm and 200 nm, 5 nm and 100 nm or 5 nm and 50 nm. In still further aspects, the particles may be nanorods having an average length of between about 5 nm and 100 nm; 10 nm and 50 nm or 10 nm and 30 nm and/or having an average width of between about 1 nm and 50 nm; 1 nm and 20 nm or 2 nm and 10 nm. In further aspects, the particles may be colloids having an average diameter between about 5 nm and 100 nm; 10 nm and 80 nm or 20 nm and 50 nm. The size and shape of the particle may be selected to achieve the desire route of clearance from the body and method of uptake by the target tumor or tumor cells.

In further aspects, the high-Z particles are administered to a subject one or more times (e.g., 2, 3, 4, 5, 6 or more times). In further aspects, the high-Z particles are administered systemically into a blood vessel, intraperitoneally, into the lymphatic system, or intratumorally. In further aspects, a de-aggregation agent can be administered to a subject one or more times (e.g., 2, 3, 4, 5, 6 or more times). In still further aspects, the target cells are irradiated within the subject. In other aspects, the target cells are irradiated extracorporeally (e.g., ex vivo). In certain aspects, the target cells comprise cancer cells, such a primary or metastatic cancer cells. In further aspects, the target cells are circulating tumor cells or blood cells. For example, the target cancer cells can be selected from the group consisting of: colorectal cancer cells, brain cancer cells, esophageal cancer cells, stomach cancer cells, liver cancer cells, biliary cancer cells, neuroendocrine cancer cells, thyroid cancer cells, lung cancer cells, pancreatic cancer cells, renal cancer cells, breast cancer cells, ovarian cancer cells, uterine cancer cells, squamous cancer cells, neuroendocrine cancer cells, melanoma cancer cells, prostate cancer cells, lymphoma cells, and/or sarcoma cells. The target cells may be primary or metastatic cancer cells.

In certain aspects, the targeting molecule has an affinity for a receptor expressed in cancer cells. For example, the targeting molecule can bind to a target selected from the group consisting of: human epidermal growth factor (EGF) receptor, human epidermal growth factor receptor 2, human epidermal growth factor receptor 3, human epidermal growth factor receptor 4, vascular endothelial growth factor receptors, folic acid receptor, melanocyte stimulating hormone receptor, integrin αvβ3, integrin αvβ5, transferrin receptor, interleukin receptors, lectins, insulin-like growth factor receptor, hepatocyte growth factor receptor, and basic fibroblast growth factor receptor. In further aspects, the targeting molecule is selected from the group consisting of: an antibody (or antigen binding fragments thereof); a polypeptide, a dendrimer, an aptamer, an oligomer, a small molecule (e.g., with affinity for the target receptor); and combinations thereof. In further aspects, the targeting molecule is selected from the group consisting of: cetuximab, an EGFr-binding peptide, trastuzumab, folic acid, melanocyte stimulating hormone, transferrin receptor targeted peptides and antibodies, and cyclic-RGD (or analogues of cyclic-RGD).

In further aspects, the ionizing radiation can be delivered in fractions over a period of time. In other aspects, the ionizing radiation can be delivered continuously, such as through a radiation-emitting substance implanted in or near target cells (e.g., a tumor). In some aspects, the radiation is delivered as proton or other heavy ion therapy. In further aspects, the ionizing radiation may be at one or more energy levels from 1 kV to 10 MV photons or up to 300 MeV heavy ions.

Aspects of the embodiment concern de-aggregation agents. As used herein “de-aggregation” agents refer to agents that affect the localization of the e.g. nanoparticles within a target cell, allowing such particles to disperse. For example, in some aspects, the de-aggregation agent is selected from the groups consisting of a lysosomotropic agent, an endosome acidification inhibitor, a vacuolar proton-adenosine triphosphate inhibitor, a proteasome inhibitor, a cathepsin A inhibitor, an intralysosomal proteolysis inhibitor, an intracellular vesicular swelling promoter and an inhibitor of late endosome lysosome fusion. In some aspects, the endosome acidification inhibitor is tamoxifen. In further aspects, the inhibitor of vacuolar-type proton-adenosine triphosphate is selected from among bafilomycin A, concanamycin A, concanamycin B and bafilomycin B1. In yet further aspects, the proteasome inhibitor and cathepsin inhibitor are selected from among lactacystin, chymostatin, clasto-Lactacystin β-lactone and MG-132. In some aspects, the inhibitor of late endosome lysosome fusion is selected from among phosphatidylinositol 3-phosphate kinase inhibitors, Rab-GDP-dissociation inhibitor (Rab-GDI), chloroquine, and primaquine. In still further aspects, the lysosomotropic agent is selected from among acetaminophen, diclofenac, rosuvastatin, amiodarone, chloroquine, amantadine, ammonium chloride, monensin, and nigericin. In certain aspects, the intralysosomal proteolysis inhibitor is selected from among suramin, phosphoramidon, leupeptin, E64, pepstatin, a cystatin or a bestatin. In further aspects, the intracellular vesicular swelling inducer is selected from among polyvinylpyrrolidone and dextran. In certain aspects, a de-aggregation agent does not include acetaminophen. In certain aspects, a de-aggregation agent does not include a chloroquine.

In still a further aspect, a method of the embodiments can comprise imaging the high-Z particles and the target cells. For example imaging can be ex vivo or in vivo.

In yet a further embodiment there is provided a pharmaceutical composition comprising (i) high-Z particles; and (ii) a de-aggregation agent. In preferred aspects, the high-Z particles comprise a targeting molecule that can bind to cells and a high-Z element. In still a further embodiment there is provided a composition for use in sensitizing target cells in a subject to a radiation therapy, the composition comprising (i) high-Z particles of the embodiments; and (ii) a de-aggregation agent. Likewise, in a further embodiment there is provided a composition for use in treating a subject who has previously been administered high-Z particles, the composition comprising a de-aggregation agent. In yet a further embodiment there is provided a composition for use in treating a subject who has previously been administered de-aggregation agent, the composition comprising an effective amount of high-Z particles.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore below 0.05%. Most preferred is a composition in which no amount of the specified component can be detected with standard analytical methods.

As used herein, the generic name of a pharmaceutical compound include its analogues. For example, the term “chloroquine” includes hydroxychloroquine and pharmaceutical compositions comprised thereof.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E: Synthesis and binding affinity of Cetuximab conjugated gold nanorods (cGNRs). (A) Cetuximab conjugation with gold nanorods (GNRs). Step I: Reaction of cetyl-trimethyl-ammonium bromide (CTAB)-coated GNRs with 2 mM PEG containing 4:1 ratio of (1) methoxy-PEG-thiol and (2) PEG bis Thiol to form pegylated GNRs (pGNRs) with free sulfhydryl moieties. Step II: Reaction of (3) cetuximab (13.16 μM) with (4) succinimidyl 4[N-maleimidomethyl]cyclohexane-1-carboxylate (2.9 mM) to form (5) maleimide-activated cetuximab. Step III: Reaction of (5) maleimide-activated cetuximab with free sulfhydryl moieties in pGNRs at a volume ratio of 5 μl/ml to form cetuximab-conjugated GNRs (c-GNRs). (B) Representative transmission electron microscopy (TEM) images of GNRs at different stages of conjugation. GNRs were negative stained to visualize the organic coating on the surface. The PEG and cetuximab coating on the GNR surface is seen as a halo with a thickness of ˜1.4 and 2.2 nm, respectively. (C) The overlay of dark field and fluorescence images of HCT116 colorectal cancer cell line at different post-incubation times with pGNR and cGNR shows the binding affinity of cGNR for the cell surface receptors, with significant binding occurring from 4 hrs post-incubation time. The blue fluorescence (DAPI) represents the nucleus of each cell. (D) Representative TEM images of HCT116 cells incubated with cGNRs showing different stages of internalization process—upper row: cell surface binding, internalization of surface-bound cGNRs, localization of internalized cGNRs within the endosome (M and N represent the mitochondria and nucleus, respectively); lower row: a cell with internalized cGNRs localized within an endosome/lysosome and a close-up image of clustered cGNRs within the endosome/lysosome. (E) High magnification (100×) dark field microscopy (DFM) image showing intracellular distribution of cGNRs that are primarily localized in the cytoplasm.

FIGS. 2A-2B: cGNR enhances biological effectiveness of radiation in vivo. (A) Biodistribution of pGNR and cGNR, represented as % injected dose (ID), in different organs of mice, at 24 hr post-injection time (i.v, 1 mg/kg). The insert shows the % ID plot without liver and spleen to highlight the enhanced uptake of cGNRs in tumor tissues. The data points represent the mean±SE of four different experiments. (B) Normalized tumor volume plot showing enhanced radiosensitization efficacy of cGNRs (20.8 μg of Au; 10 Gy; 250 kVp). Each data point represents the mean±SE of five animals.

FIGS. 3A-3B: Active targeting using cGNRs enhances in vitro radiation sensitivity via increased DNA damage. Clonogenic cell survival curves of HCT116 cells treated with 250 kVp radiation (A) 30 min and (B) 24 hrs post-incubation with pGNR or cGNRs (0.001% wt/vol gold). Prior to radiation, the media containing pGNR and cGNR was aspirated and fresh media was added

FIG. 4: Active targeting using cGNRs radiosensitizes cells via increased oxidative stress. NADP+/NADPH ratio, an indicator of cellular redox balance, of cells pretreated with cGNR showed ˜3-fold increase at 1 hr post-irradiation when compared to the cells pretreated with pGNR or treated with radiation alone.

FIGS. 5A-5G: Blocking endocytosis steps modulates radiosensitization by cGNRs. (A) Clonogenic survival of HN5 cells following exposure to 250 kVp radiation: Cells were pre-treated with PBS, 5 μg/ml chlorpromazine (CPZ), 100 nM bafilomycin A1 (BAF-A1), 1 μM lactacystin (LAC), or 5 μM chloroquine (CHQ) for 1 hr and incubated with cGNR for 24 hr followed by exposure to 0, 2, 4, and 6 Gy radiation. The radiation dose enhancement factor (DEF) at 10% cell survival was cGNR—1.08, CPZ+cGNR—1.005, BAF-A1+cGNR—1.19, LAC+cGNR—1.16, and CHQ+cGNR—1.20. (B) Intracellular localization cGNR in HN5 cells imaged by TEM 24 hrs after treatment with cGNRs in the presence of (a) PBS, (b) CPZ, (c) BAF-A1, (d) LAC or (e) CHQ for 1 hr. Left panel, 5,000×; middle panel 50,000×, shows the intracellular localization of cGNR; right panel, 100,000×, showing clustered cGNRs in endosomes after cGNR treatment but unclustered cGNRs after cGNR treatment after exposure to BAF-A1, LAC and CHQ. CPZ blocked internalization of cGNRs. (C) Time course of γ-H2AX foci formation and resolution following treatment with cGNRs and radiation in the absence or presence of specific inhibitors of internalization (foci averaged across 60 cells) demonstrating increased foci formation soon after radiation and cGNR treatment when pretreated with BAF-A1, LAC and CHQ but not CPZ. (D) Total number of micronuclei per 1000 binucleated cells treated with cGNRs and radiation in the absence or presence of specific inhibitors of internalization supporting the enhancement in radiosensitization when cells are pretreated with BAF-A1, LAC and CHQ. (E) Reactive oxygen species measured by CellROX assay kit following treatment of HN5 cells with 2 Gy radiation in the absence or presence of specific inhibitors of internalization with and without cGNRs. This demonstrates increased oxidative stress with radiation and cGNRs compared to radiation alone and an even greater enhancement of oxidative stress when cells are pretreated with BAF-A1, LAC and CHQ. (F) Elemental gold content of cells analyzed by inductively coupled plasma mass spectrometry. Lack of internalization of pGNRs and CPZ-pretreated cGNRs results in negligible quantities of gold in cells whereas once internalized, the amount of gold within cells is high in cells treated with cGNRs but no different between cells treated without or with BAF-A1, LAC and CHQ. (G) Tumor regrowth delay of HN5 xenografts treated radiation (10 Gy, 6 MV, 1.5 cm bolus over tumors) 24 hrs following intravenous administration of 40 μg cGNRs demonstrating statistically significant increase in time to tumor volume quadrupling when CHQ was administered 6 hrs and 24 hrs before radiation (p=0.02).

FIGS. 6A-6D: Intracellular localization of cGNRs enhances microscopic dose enhancement. Isodose plots of radiation dose enhancement around cGNRs upon irradiation with (A) 250 kVp and, (B) 6 MV photon beams. (C) Dose area histogram showing the radiation dose enhancement across the nucleus, for 250 kVp and 6 MV beams. (D) Cartoon illustrating the mechanism of cGNR mediated radiosensitization and the effect of the de-aggregation agents described herein.

FIG. 7: Plasmon resonance spectrum of GNRs during different stages of bioconjugation shows distinct transverse and longitudinal peaks around 510 and 780 nm. Prior to conjugation, CTAB-GNRs demonstrated a resonance peak at 782 nm. Pegylation with 4:1 ratio of mPEG-SH and SH-PEG-SH and subsequent coating with cetuximab resulted in a spectral blue shift of longitudinal resonance peak to 778 and 776 nm, respectively.

FIG. 8: Zeta potential ζ of GNRs at different stages of conjugation. CTAB-GNRs demonstrated a strong positive ζ of 47±3.6 mV. Upon pegylation with 4:1 ratio of mPEG-SH and SH-PEG-SH and subsequent conjugation with cetuximab, the ζ values shifted towards negative values of −5±1.0 mV and −10±1.3 mV, respectively, demonstrating efficient replacement of CTAB. The ζ of HCT116 cells, measured as −13±0.4 mV, shown in this figure suggests that the cellular uptake of cGNRs is not due to electrostatic binding but possibly via receptor-mediated endocytosis.

FIG. 9: Tumor regrowth delay of HCT116 xenografts treated with radiation (10 Gy, 6 MV, 1.5 cm bolus over tumors) 24 hrs following intravenous administration of cGNRs; pGNRs; non-specific antibody conjugated GNRs (iGNRs); or pGNRs and an equivalent concentration of cetuximab (C225). A significant delay in time to tumor volume doubling was achieved only when cGNRs treatment was combined with radiation indicating that the observed radiosensitization is not due to a non-specific effect of decorating GNRs with an antibody or a non-specific interaction between GNRs and cetuximab.

FIG. 10A-10C: Number of micronuclei per 1000 binucleated cells broken down as (A) one, (B) two, or (C) multiple. Cells were treated with cGNRs and radiation in the absence or presence of specific inhibitors of internalization. Across all methods of examining the number of micronuclei per 1000 binucleated cells, there was a significant increase in radiation-induced micronuclei formation when cells exposed to cGNRs for 24 hrs prior to irradiation were pretreated for 1 hr with 100 nM bafilomycin A1 (BAF-A1), 1 μM lactacystin (LAC), and 5 μM chloroquine (CHQ) but not 5 μg/ml chlorpromazine (CPZ).

FIG. 11A-11C: (A) Clonogenic survival assay: HN5 cells were seeded in petri dishes for 48 h. Later, at 70% confluence cells were incubated with PBS, pGNR (OD=0.5), or cGNR (OD=0.5) for 24 h. Cells were irradiated with 2, 4, and 6 Gy of X-Ray (250 kVP, 15 mA). After irradiation known number of HN5 cells were seeded in six well plates and incubated for 14 days to count colonies. Radiation Dose Enhancement Factor was calculated 1.04 and 1.34 for pGNR and cGNR respectively. (B) Internalization of gold nanorods: 20 k HN5 cells were seeded per well in 8 chambered slides. When cells were 70% confluent, cGNR or pGNR (OD 0.5) were added for 24 h. Cell were fixed in 4% paraformaldehyde and 70% ethanol, and counterstained with Hoechst 33342. After washing, slides were mounted with progold mounting media. Internalization of gold nonorods was observed at 100× by darkfield microscopy. (C) Hyperspectral analysis of internalization of gold nanorods: HN5 cells were seeded in chamber slides. At 70% confluence cells were treated with 0.5 OD of cGNR and pGNR for 24 h. Later cells were fixed in 4% paraformaldehyde and 70% ethanol. After that slides were counterstained with Hoechst 33342 washed and mounted with progold mounting media. The hyperspectral image analysis was carried out at 700, 725, 750, 775, and 800 nm. The representative images show the analysis at 775 nm. Bottom panel graphs show plots of intensity (y-axis) vs. wavelength (x-axis).

FIG. 12A-C: Spherical gold nanospheres (GNS) were conjugated to either PEG (pGNS), non-specific antibody (iGNS) or Cetuximab (cGNS). FIG. 12A, the absorbance profiles for the particles. FIG. 12B is a graph showing particle uptake into HN5 head and neck cancer cells. In graph of FIG. 12C the particles were evaluated for their ability to sensitize FINS cells to radiation in clonogenic assays where cultured cells were exposed to varying doses of radiation 24 hours after treatment with 0.5 OD GNS.

### DESCRIPTION OF ILLUSTRATIVE EMBODIMENTS

Radiation dose enhancement within tumors can be achieved with nanoparticles made of high atomic number (Z) elements such as gold. Current methods require large quantities of gold, kilovoltage radiotherapy and a short interval between nanoparticle administration and radiotherapy. Embodiments of the present invention provide methods for radiosensitizing target cells and tumors using nanoparticles. In particular, methods of radiosensitization comprise the administration of high-Z nanoparticles, such as gold nanorods (GNRs) or colloidal gold nanoparticles (GNPs) conjugated to a cell targeting agents (e.g., tumor-targeting antibodies). The radiosensitization is mediated, in part, by internalization of GNRs within cells and the consequent increases in oxidative stress and DNA damage. In certain embodiments, internalization-dependent radiosensitization can be enhanced by de-aggregation agents, such as pharmacological inhibitors of endocytosis that cause vacuolar acidification and disaggregation of particles within endosomes. By the combined use of high-Z particles and de-aggregation agents significant radiosensitization can be achieved using clinically relevant amounts of particles.

In particular, experiments disclosed herein use cylindrical shaped GNRs or spherical GNPs and an active targeting strategy for tumor cell-specific delivery and present the mechanism of action leading to enhanced radiosensitization. In an exemplary method, GNRs and GNPs were conjugated to cetuximab, a monoclonal antibody targeting the epidermal growth factor receptor (EGFR) which is overexpressed in many cancer cells, and evaluated in vitro or in vivo. The results disclosed herein demonstrate that active targeting resulted in enhanced radiosensitization with clinically-relevant gold concentrations and 6 MV beams. Mechanistic investigations suggest that the receptor-mediated endocytosis of GNRs enhances GNR-mediated radiosensitization. It was also discovered that the internalization can be leveraged to further enhance the radiosensitization effect with a pharmaceutical agent by inducing intracellular disaggregation of particles. A Monte Carlo-based computational approach estimated the spatial variations of intracellular microscopic dose enhancement due to internalized GNRs and provided a basis for comparison of comparable effects observed with 250 kVp and 6 MV photon beams.

Thus, the methods disclosed herein offer the first translatable strategy to radiosensitize tumors with clinically-achievable gold concentrations. In some embodiments, the radiosensitization effect can be amplified using a de-aggregation agent such as chloroquine.

### I. DEFINITIONS

As used herein, the term “radiosensitize,” when used in reference to a tumor or a tumor cell, means to increase susceptibility of the tumor or tumor cell to the effects of radiation. It is recognized that the term “radiosensitize” is used in a comparative sense and, with regard to the present invention, indicates that the radiation dose to reduce the severity of a cancer in a subject that has been administered nanoparticles as disclosed herein is less than the radiation dose that would have been required if the subject had not been administered nanoparticles. In contrast, the term “radioresistant” means that a cell is relatively refractory to the effects of radiation.

The term “radiation” is a process in which energetic particles or energy or waves travel through a medium or space.

The term “ionizing radiation” refers to radiation comprising particles or photons that have sufficient energy or can produce sufficient energy via nuclear interactions to produce ionization (i.e., gain or loss of electrons). An exemplary ionizing radiation is an x-radiation. Means for delivering x-radiation to a target tissue or cell are well known in the art. The amount of ionizing radiation needed in a given cell generally depends on the nature of that cell. Means for determining an effective amount of radiation are well known in the art. Dosage ranges for x-rays range from daily doses of 50 to 200 cGy for prolonged periods of time (3 to 8 weeks), to single or a small number (3-5) doses of 500 to 2000 cGy. Dosage ranges for radioisotopes vary widely, and depend on the half-life of the isotope, the strength and type of radiation emitted, and the uptake by the neoplastic cells. In general, however, dose ranges for heavy ions are similar to those for x-rays.

The phrase “effective amount” means a dosage of a drug or agent sufficient to produce a desired result. The desired result can be subjective or objective improvement in the recipient of the dosage, a decrease in tumor size, a decrease in the rate of growth of cancer cells, a decrease in metastasis, or any combination of the above.

Used herein, the term “an effective dose” of ionizing radiation means a dose of ionizing radiation that produces an increase in cell damage or death when given in conjunction with the nanoparticles of the invention, optionally further combined with a chemotherapeutic agent.

The term “tumor cell” or “cancer cell” denotes a cell that demonstrates inappropriate, unregulated proliferation. A “human” tumor is comprised of cells that have human chromosomes. Such tumors include those in a human patient, and tumors resulting from the introduction into a non-human host animal of a malignant cell line having human chromosomes. However, “tumor cell” or “cancer cell” may also denote cells of non-human animals.

As used herein the term “targeting molecule” or “targeting moiety” refers to any suitable targeting moiety that can be either chemically conjugated to, or directly complexed with, the nanoparticles provided herein. The term can also refer to a functional group that serves to target or direct a therapeutic agent or anti-cancer agent to a particular location, cell type, diseased tissue, or association. In general, a “targeting ligand” can be directed against a biomarker. The terms “targeting ligand” and “targeting moiety” are used interchangeably throughout.

As used herein, the term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. In exemplary embodiments, antibodies used with the methods and compositions described herein are derivatives of the IgG class. The term antibody also refers to antigen-binding antibody fragments. Examples of such antibody fragments include, but are not limited to, Fab, Fab′, F(ab′)2, scFv, Fv, dsFv diabody, and Fd fragments. Antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 10 amino acids and more typically will comprise at least about 200 amino acids.

A “chemotherapeutic” drug as used herein refers to those drugs commonly used in the treatment of cancer. These agents act through an apoptotic mechanism of cell death. Each of the drugs can differ in the mechanism by which the cells enter apoptosis.

As used herein, “inhibit” means to reduce by a measurable amount, or to prevent entirely.

As used herein, “to treat” means to inhibit or block at least one symptom that characterizes a pathologic condition, in a mammal threatened by, or afflicted with, the condition.

As used herein, the term “nanoparticle” refers to any particle having dimensions of less than 1000 nanometers (nm). Nanoparticles can be optically or magnetically detectable. In some embodiments, intrinsically fluorescent or luminescent nanoparticles, nanoparticles that comprise fluorescent or luminescent moieties, plasmon resonant nanoparticles, and magnetic nanoparticles are among the detectable nanoparticles that can be used in various embodiments. In general, the nanoparticles should have dimensions small enough to allow their uptake by eukaryotic cells. Typically, the nanoparticles have a longest straight dimension (e.g., diameter) of 200 nm or less. In some embodiments, the nanoparticles have a diameter of 100 nm or less. Smaller nanoparticles, such as having diameters of 50 nm or less, such as about 1 nm to about 30 nm or about 1 nm to about 5 nm, are used in some embodiments. The size and shape of the particle may be selected to achieve the desired circulation, clearance or uptake kinetics. Depending on the shape of the nanoparticle, the size relates to the diameter or length of the respective structure.

In various embodiments, the size is the mean particle size. The nanoparticle may be comprised or one or more high-Z elements. A gold nanoparticle may be selected from the group consisting of a gold nanosphere, a gold nanorod, a gold nanotube, a gold nanoshell, a gold nanodot, a gold nanowire, and a gold nano triangle.

### II. EMBODIMENTS OF THE PRESENT INVENTION

A. Nanoparticles

Nanoparticles comprised of high atomic weight (high-Z) elements are allowed to specifically accumulate in the target of radiation therapy, providing a localized dose enhancement as a result the interaction of the high atomic number (Z) element with the incident radiation. Metals used to form the nanoparticles disclosed herein include, but are not limited to, gold, silver, iron, cobalt, zinc, cadmium, nickel, gadolinium, chromium, copper, manganese, palladium, tin, and alloys and/or oxides thereof. The nanoparticles can be in the form of nanorods, nanoshells, gold colloids, iron colloids, iron oxide, gadolinium colloids, nanocages, or nanoprisms. Key considerations in the selection of the particle geometry include the intravenous circulation kinetics and the particle uptake by the tumor and target cells. For example, gold is particularly useful because of its biocompatibility.

Currently, across a broad spectrum of gold nanoparticle geometries that are readily synthesized, gold nanorods (GNRs) offer a few unique characteristics. They are highly stable, cylindrical, solid gold nanoparticles with sufficient gold content to elicit potent radiation dose enhancement. They also absorb strongly in the near infrared (NIR) wavelengths to generate intense heat by photothermal activation of their surface plasmons (Huang, et al. 2007). When administered systemically in mice via the tail vein GNRs accumulate passively in tumors via the enhanced permeability and retention effect and, being strong metallic conductors, deliver this photothermal energy directly and efficiently to tumors. Tumor uptake can be enhanced by evading reticuloendothelial capture via surface coating with polyethylene glycol (PEG) or due to their shape (Caliceti and Veronese 2003; Mitragotri and Lahann 2009). The PEG coating also allows further functionalization with peptides, antibodies and oligonucleotides decorating the surface as well. In vitro, the cylindrical shape of GNRs enhances their internalization into cells. (Huff, et al. 2007; Hauck, et al. 2008). In vivo, their cylindrical shape leads to their marginalization to the vascular endothelial surface rather than the center of a parabolic advancing front of blood within a blood vessel, allowing greater extravasation from a blood vessel exhibiting laminar flow and more so from a vessel exhibiting turbulent flow (Ferrari et al., 2009). Lastly, as with other GNPs, favorable characteristics of GNRs used in clinical applications are the lack of biochemical reactivity, inertness, and clinical safety record of gold used for decades in the treatment of arthritis. Although GNRs are efficient photothermal activators, this property is not directly invoked in all embodiments of this method. Nonetheless, this property could certainly be exploited for specific clinical scenarios such as during intraoperative radiation therapy or for triggered release of chemotoxic agents.

In one embodiment, the NP is spherical. In another embodiment the NP is rod-shaped. In other embodiments, the NP is either triangular, ellipsoidal or cubic in shape. In another embodiment, the high-Z element may be contained in a liposome or micelle.

In one embodiment, the NP is less than 400 nm and greater than 8 nm along its longest dimension. In another embodiment, the NP is preferably greater than 10 nm and less than 200 nm along its longest dimension. In another embodiment, the NP is preferably greater than 20 nm and less than 100 nm along its longest dimension.

The GNRs for use in the compositions and methods of the embodiments have a length of from about 10 to about 100 nm, inclusive, and including all integers there between. In one embodiment, the GNRs have an average length of from 70-75 nm. The GNRs have a diameter of from 5 to 45 nm inclusive, and including all integers there between. In one embodiment, the GNRs have an average diameter of 25-30 nm. In some aspects, the GNPs for use in the compositions and methods of the embodiments have a diameter of from about 5 to about 100 nm, inclusive, and including all integers there between. In one embodiment, the GNPs have an average diameter of from 20-50 nm, and including all integers there between. In other embodiments, the nanoparticles can have an average diameter of about 10 nm or less. Nanoparticles with an average or nominal diameter of about 5 nm or less can be readily cleared from the subject by reticular endothelium system after delivery of the hydrophobic therapeutic agent to the targeted cell or tissue.

In one embodiment, the NP is comprised of at least 50% by mass of a high-Z element. In other embodiments, the NPs are comprised of at least 60%, 70%, 80%, 90%, 95% or 99% by mass of a high-Z element.

The GNRs can be pure gold, or may be from 90% to 99%, inclusive, including all integers there between, pure gold. In various embodiments, the GNRs may contain up to 1% silver on their surfaces, and may contain cetyltrimethylammonium bromide (CTAB). In this regard, GNRs can be made by any suitable method. For example, electrochemical synthesis in solution, membrane templating, photochemical synthesis, microwave synthesis, and seed mediated growth are all suitable and non-limiting examples of methods of making the GNRs. In one embodiment, the gold nanorods are made using the seed-mediated growth method in cetyltrimethylammonium bromide (CTAB).

Several methods are available for the manufacture of gold nanorods including electrolytic, chemical reduction, and photoreduction processes. In the electrolytic method, a solution containing a cationic surfactant is electrolyzed with constant current, and gold clusters leached from a gold plate at the anode. In one chemical reduction method, NaBH₄ reduces chlorauric acid and gold nanoparticles are generated. These gold nanoparticles act as “seed particles” and growing them in solution results in gold nanorods. The length of the gold nanorods generated is influenced by the ratio of the “seed particles” to chlorauric acid in the growth solution. With the chemical reduction method, it is typically possible to generate longer gold nanorods relative to electrolytic methods. With the photo-reduction method, chlorauric acid is added to substantially the same solution as that in the electrolytic method, and ultraviolet irradiation effects the reduction of chlorauric acid. It is generally possible to control the length of the gold nanorods by the irradiation time. Likewise, several methods are available for the manufacture of spherical gold nanoparticles (see, for example, Graber et al. 1995, which is incorporated herein by reference).

In one embodiment, the NP surface is conjugated with a polymer to increase circulation time in the blood stream. In one embodiment, the polymer is preferably a polyethylene glycol. The polymer coating can provide a protective shell that increases the hydrophilicity of the nanoparticle and biocompatibility of the targeted nanoparticle conjugates and postpone and/or delay clearance of the targeted nanoparticle conjugates after delivery to the subject by reticular-endothelium system. The polymer coating also acts as an amphiphilic reservoir that can adsorb and stabilize hydrophobic therapeutic agents in aqueous medium and/or blood of the subject without the need to modify the structure of the therapeutic agent. The adsorption and stabilization of the hydrophobic therapeutic agent allows the hydrophobic therapeutic agent to be delivered to the targeted cell or tissue by the targeted nanoparticle conjugates and minimizes side effects.

The polymers used to coat the nanoparticles can include natural proteins, such as bovine serum albumin (BSA), biocompatible hydrophilic polymers, such as polyethylene glycol (PEG) or a PEG derivative, phospholipid-(PEG), lipids, and carbohydrates, such as dextran. Coatings of polymer may be applied or assembled in a variety of ways, such as by dipping, using a layer-by-layer technique, by self-assembly, or conjugation. Self-assembly refers to a process of spontaneous assembly of a higher order structure that relies on the natural attraction of the components of the higher order structure (e.g., molecules) for each other. Self-assembly typically occurs through random movements of the molecules and formation of bonds based on size, shape, composition, or chemical properties.

In one embodiment, the polymer coating can include polyethylene glycol (PEG). The PEG can be a hetero-bifunctional PEG, such as COOH-PEG-SH (MW 3000), and/or a monofunctional PEG, such as PEG-SH (MW 5000), that can readily bind to the nanoparticle to coat the nanoparticle. In some embodiments, the nanoparticle can be coated with a mixture of hetero-bifunctional PEG, such as COOH-PEG-SH (MW 3000), and monofunctional PEG, such as PEG-SH (MW 5000). The mixture can range in percent composition of hetero-bifunctional PEG to monofunctional PEG of about 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, and 99:1 respectively.

B. Targeting of Particles to Target Cells

Initial Monte Carlo studies of GNP-based radiation dose enhancement demonstrated that the macroscopic (or average) tumor dose enhancement depends on the gold concentration within the tumor and the photon beam quality, ranging from several hundred percent for diagnostic x-rays to a few percent for typical clinical megavoltage photon beams. (Cho, 2005) For instance, the dose enhancement for a superficial tumor treated with 140 kVp x-rays could be at least a factor of 2 at a gold concentration of 7 mg Au/g tumor (7 parts per thousand). As predicted by subsequent Monte Carlo models (Cho, et al. 2009), to maximize the dose enhancement, the ideal source would have a dominant energy spectrum just above the K-edge of gold (80.7 keV) like ¹⁶⁹Yb (half-life=32.0 days, intensity-weighted average gamma ray energy 93 keV) brachytherapy source or the L-edge of gold (about 10 keV) like the classical brachytherapy sources ¹⁰³Pd and ¹²⁵I or the low energy 50 kVp x-ray source. In all of these instances, the efficacy of treatment depends upon the sustained presence of GNPs within the tumor but not within the adjacent normal tissue. The selective uptake of nanoparticles by the tumor can be achieved by selecting size and shape and appropriate targeting moieties. Thus, the high-Z particles disclosed herein can be actively targeted to the targeted tissue or targeted population of cells. The target cells can be tumor cells, endothelial cells, tumor stromal cells, circulating tumor cells and/or blood cells.

The selection of a targeting ligand is dependent on the targeting objectives. In general, nanoparticles enter a tumor through the enhanced permeability and retention effect. Once in the tumor, the targeting ligand may be used to increase retention in the tumor and reduce lymphatic clearance. Alternatively, the targeting ligand may be selected to result in internalization of the nanoparticles within the tumor cell, which generally requires binding or affinity with a cell surface molecule. In certain cases, if the tumor vasculature is the target of therapy, the targeting ligand may be chosen to result in binding or affinity with a cell surface ligand, and may be selected to result in internalization within the endothelial cell. Alternatively, the nanoparticles may be designed to enhance internalization within the tumor cell, which may be affected by the shape or surface charge of the particle.

The targeting moiety can include any molecule, or complex of molecules, which is/are capable of targeting, interacting with, coupling with, and/or binding to an intracellular, cell surface, or extracellular biomarker of a cell or tissue. The biomarker can include, for example, a cellular protease, a kinase, a protein, a cell surface receptor, a lipid, and/or fatty acid. Other examples of biomarkers that the targeting moieties can target, interact with, couple with, and/or bind to include molecules associated with a particular disease. For example, the biomarkers can include cell surface receptors implicated in cancer development, such as epidermal growth factor receptor and transferrin receptor. The targeting moieties can interact with the biomarkers through, for example, non-covalent binding, covalent binding, hydrogen binding, van der Waals forces, ionic bonds, hydrophobic interactions, electrostatic interaction, and/or combinations thereof.

The targeting moieties can include, but are not limited to, synthetic compounds, natural compounds or products, macromolecular entities, bioengineered molecules (e.g., polypeptides, lipids, polynucleotides, antibodies, antibody fragments), and small entities (e.g., small molecules, neurotransmitters, substrates, ligands, hormones and elemental compounds).

In one example, the targeting moiety can include an antibody, such as a monoclonal antibody, a polyclonal antibody, or a humanized antibody. The antibody can include Fv fragments, single chain Fv (scFv) fragments, Fab′ fragments, F(ab′)2 fragments, single domain antibodies, camelized antibodies and other antibody fragments. The antibody can also include multivalent versions of the foregoing antibodies or fragments thereof including monospecific or bispecific antibodies, such as disulfide stabilized Fv fragments, scFv tandems ((scFv)2 fragments), diabodies, tribodies or tetrabodies, which typically are covalently linked or otherwise stabilized (i.e., leucine zipper or helix stabilized) scFv fragments; and receptor molecules, which naturally interact with a desired target molecule.

With respect to cell surface molecules that may be targets, it is known that certain tumor cell surface targets may result in internalization of the nanoparticle within the cell. Additionally, internalization may be induced or affected by the specific nanoparticle chosen. Among the tumor and tumor-vasculature related targets are the epidermal growth factor receptor, human epidermal growth factor receptor 2, human epidermal growth factor receptor 3, human epidermal growth factor receptor 4, vascular endothelial growth factor receptor, the folate receptor, the melanocyte stimulating hormone receptor, the vascular endothelial growth factor receptors, and the integrins, transferrin, interleukins, insulin-like growth factor receptor, hepatocyte growth factor receptor, and basic fibroblast growth factor receptor. The targeting ligands to these receptors include antibodies (or portions thereof), peptides, aptamers, proteins, and other molecules. For example, targeting ligands can be cetuximab, herceptin, folic acid, melanocyte stimulating hormone, cyclic-RGD, and an analogue to cyclic-RGD.

Among a wide array of tumor-specific biomarkers, the epidermal growth factor receptor (EGFR) is increasingly viewed as a viable therapeutic target because it is ubiquitously over-expressed in a wide variety of cancers and drives their unchecked growth and proliferation, invasiveness, angiogenic and metastatic potential, and resistance to traditional cancer therapies. Specific targeting of this receptor using a humanized monoclonal antibody (cetuximab) improves patient survival in a variety of clinical situations (Goldberg, 2005). In particular, the efficacy of combination of cetuximab and radiation in head and neck cancer led to the first and only approval of a targeted therapy combination with radiation therapy (Bonner et al. 2006).

Preparation of antibodies can be accomplished by any number of methods for generating antibodies. These methods typically include the step of immunization of animals, such as mice or rabbits, with a desired immunogen (e.g., a desired target molecule or fragment thereof). Once the mammals have been immunized, and boosted one or more times with the desired immunogen(s), antibody-producing hybridomas may be prepared and screened according to well known methods. See, for example, Kuby, Janis, Immunology, Third Edition, pp. 131-139, W.H. Freeman & Co. (1997), for a general overview of monoclonal antibody production, that portion of which is incorporated herein by reference.

In vitro methods that combine antibody recognition and phage display techniques can also be used to allow one to amplify and select antibodies with very specific binding capabilities. See, for example, Holt, L. J. et al., “The Use of Recombinant Antibodies in Proteomics,” Current Opinion in Biotechnology, 2000, 11:445-449, incorporated herein by reference. These methods typically are much less cumbersome than preparation of hybridomas by traditional monoclonal antibody preparation methods.

Non-invasive radiation response modulation may be achieved using targeting molecules and high-Z particles, such as the cetuximab-conjugated GNRs, that can be widely applied as a class solution across multiple tumor types over-expressing the target receptor. For example, EGFR is widely expressed in several cancers. Alternatively, the folate receptor is expressed on many cancer cells, and the particle may be targeted using folic acid. Alternatively, the melanocyte stimulating hormone receptor is expressed on melanoma cells, and the particle may be targeted using the melanocyte stimulating hormone. Alternatively, the integrin alpha-v beta-3 is expressed on the endothelial cells of tumors and on certain cancer cells, and the particle may be targeted using the peptide cyclic-RGD or one of its analogues. Alternatively, the HER-2 receptor is expressed on certain breast and ovarian cancers, and the particle may be targeted with an anti-HER-2 antibody or fragment. Similar results may be achieved using any or a combination of targets.

The targeting moiety is usually attached in a manner that allows access to the receptor on the target cell. For example, this may be accomplished with the attachment of the targeting molecule through a bi-functional polyethylene glycol chain. Steric hindrance of the targeting molecule should be avoided by proper selection of the linking method.

Radiation dose enhancement is increased when the high-Z NP is closest to the DNA of the target cell. This is first accomplished by selection of a targeting molecule or NP with properties (such as shape or charge) that are internalized by the cell. Additional targeting molecules may be conjugated to the particle that will allow transit through the nuclear membrane. Alternatively, this may be accomplished through the use of a single or multiple targeting molecules.

C. Radiotherapy

Radiation therapy is a conventional method for treating cancer. In particular, radiotherapy is useful in cases where the tumor is relatively localized and not excessively large, or where surgical excision of the tumor is contraindicated due, for example, to the location of the tumor. Radiation therapy is a preferred method of treating, for example, prostate cancer and brain tumors. Common sources of radiotherapy include kilovoltage x-ray sources, LINACs, and particle radiotherapy. The skilled artisan would know the appropriate dosages, treatment schedules and radiation sources to use for treating a particular cancer.

Various factors can limit the usefulness of radiotherapy. Ultimately, however, the success of radiotherapy is limited due to unacceptable patient morbidity that occurs as a result of consequent irradiation of normal tissue in the radiation field. In particular, exposure of rapidly renewing tissues, including, bone marrow, small intestine and skin, to radiation can lead to unacceptable patient morbidity. However, slowly proliferating tissues, including nervous tissue, also can be damaged irreversibly if exposed to an excessively high dose of radiation.

A current challenge in radiotherapy is to provide a lethal dose only to a tumor within the tolerance of essential normal tissue. Devices like accelerator-based megavolt x-ray generators, tomography machines, stereotactic radiotherapy systems and intensity modulated radiation therapy systems are not sufficient to treat cancers because doses sufficient to kill the cancer are often equally or more likely to damage adjacent normal tissue, thus limiting the ability to safely treat the patient. The nanoparticle compositions and methods disclosed herein serve as tumor-specific radiosensitizers to compensate for the insufficiency of equipment-based treatment.

In some embodiments, high-Z nanoparticles are administered in combination with radiotherapy for an enhanced radiation effect. The radiotherapy can be ionizing radiation administered at one time or as fractions over a period of time. Administration of radiotherapy as fractionated doses over a period of time can provide advantages over administration of a single large dose. In particular, fractionated doses of radiation are useful if the cells in the normal tissue in the radiation field can repair radiation induced damage faster or more efficiently than the tumor cells in the radiation field. In this case, fractionated doses can preferentially allow repair of the normal cells as compared to the tumor cells. In addition, tumors generally have relatively hypoxic regions that are less susceptible to radiation damage. Fractionated radiation doses also can permit reoxygenation to occur in such regions, due to sloughing off of tumor cells killed by previous doses, thus improving the effectiveness of subsequent radiation doses. Additionally, the high-Z nanoparticles can be administered one or more times, such as 2, 3, 4, 5, or 6 times. The targeted tissue or targeted population can be irradiated in vivo (i.e., within the animal) or extracorporeally.

The present disclosure relates to the design, manufacturing, and use of high-Z particles to enhance the effects of ionizing radiation. The localization of a high-Z particle near the nucleus of a target cell may enhance the effect of ionizing radiation and increase DNA strand damage, resulting in a therapeutic benefit. In particular, the use of a targeting molecule to enable cellular uptake by the target cells (tumor cells or endothelial cells proximate to the tumor) will enhance the dose effect. In one embodiment, gold nanoparticles are the high-Z particles because of their biocompatibility. However, other high-Z elements may also be used. Alternatively, the nanoparticle may be chosen with properties that will result in cellular uptake in the tumor. These properties may include surface charge or shape.

In one embodiment, the ionizing radiation is directed to a target cell or tissue using external beam radiation, including intensity modulation or conforming beam methods. In another embodiment, the ionizing radiation is delivered intratumorally by brachytherapy seeds or other methods. In a separate embodiment, the source of radiation may be protons or other charged particles.

In one embodiment, the energy of the radiation source is above the K-edge of the high-Z NP. In another embodiment, the energy of the radiation source is not selected based on the K-edge of the high-Z NP.

The targeted particle that accumulates within the tumor is expected to persist longer in the tumor because the targeting molecule enables cellular uptake. As a result, the dose enhancement effect may be achieved through a series of sequential irradiations, a practice common in radiation therapy today. Alternatively, subsequent doses of the targeted particle may be administered during the course of radiation to maintain a dose enhancement effect during the course of radiation treatments.

In another embodiment, the target is exposed to ionizing radiation in a continuous flow extracorporeal device. For example, circulating tumor cells may be targeted by the high-Z particle by injecting the particle into the blood stream, allowing a time delay for uptake by the tumor cells. The blood may then be circulated through an extracorporeal device and exposed to ionizing radiation in the device, and then the blood reinjected into the blood stream, all in a continuous loop.

In the methods disclosed herein, the particle dose is several orders of magnitude less than the doses previously used to achieve a radiation dose enhancement. This is principally the result of timing the irradiation to match the uptake of the particle in the tumor and using a targeting molecule free of steric hindrance to result in longer tumor retention as well as cellular uptake. In one embodiment, the particle dose that accumulates in the tumor is less than 0.05% by mass of the target tissue. In one embodiment the particle dose administered parenterally in each administration is less than 0.05% by mass of the animal mass.

D. Enhanced Radiosensitization

Endocytosis is a form of active transport in which a cell transports molecules such as proteins into the cell by engulfing them in an energy-using process. Receptor-mediated endocytosis also known as clathrin-dependent endocytosis is a process by which cell absorb metabolites, hormones, and other proteins by the inward budding of plasma membrane vesicles containing proteins with receptor sites specific to the molecules being absorbed. Receptor-mediated endocytosis of GNRs or GNPs plays an important role in GNR-mediated and GNP-mediated radiosensitization.

The endocytosis of nanoparticles results in aggregation of the nanoparticles within endosomes or other vesicles within the cell. One key finding disclosed herein is that reduction of this aggregation will increase the radiosensitization within the target cell. Accordingly, the methods of radiosensitization disclosed herein can combine administration of high-Z nanoparticles with at least one inhibitor of endocytosis to enhance the radiation effect. Administration of the nanoparticles can be combined with any inhibitor of endocytosis known in the art. Inhibitors of endocytosis include, but are not limited to, chloroquine, chloropromazine, phenylarsine oxide, monensin, phenotiazines, methyl-β-cyclodextrin, filipin, cytochalasin D, latrunculin, amiloride, and dynasore. One exemplary method combines the administration of cetuximab-conjugated gold nanorods with chloroquine.

In the methods of the present invention, the actual dosage of the endocytosis inhibitor employed will depend on a variety of factors including the type and severity of cancer being treated, and the additive or synergistic treatment effects of the nanoparticles and endocytosis inhibitor.

E. Treatment Methods

Tumors that can be suitably treated with the methods of the present invention include, but are not limited to, tumors of the brain (e.g., glioblastomas, medulloblastoma, astrocytoma, oligodendroglioma, ependymomas), lung, liver, spleen, kidney, lymph node, small intestine, pancreas, blood cells, colon, stomach, breast, endometrium, prostate, testicle, ovary, skin, head and neck, esophagus, bone marrow, blood and other tissue. The tumor may be distinguished as metastatic and non-metastatic.

The individual to which the composition of the invention is administered is a mammal. In one embodiment, the mammal is a human.

Any suitable means for delivering radiation to a tissue may be employed in the methods disclosed herein, in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

If desired, compositions comprising the nanoparticles may be prepared by mixing with any suitable pharmaceutically acceptable carriers, excipients and/or stabilizers. Some examples of compositions suitable for mixing with nanoparticles can be found in: Remington: The Science and Practice of Pharmacy (2005) 21st Edition, Philadelphia, Pa. Lippincott Williams & Wilkins.

It is expected that the compositions of the invention can be administered to an individual using any available method and route, including oral, parenteral, subcutaneous, intraperitoneal, and intrapulmonary injections. Parenteral infusions include intramuscular, intravenous, intraarterial, intratumoral, intraperitoneal, and subcutaneous administration. In an exemplary method, the nanoparticles are administered intravenously. In other embodiments, the nanoparticles are administered into the lymphatic system. In another embodiment, the nanoparticles are directly injected into the tumor.

Administration of the compositions disclosed herein can be performed in conjunction with conventional therapies that are intended to treat a disease or disorder. The composition could be administered prior to, concurrently, or subsequent to conventional therapies for the diseases or disorders. For example, a composition of the invention could be administered in conjunction with anti-cancer therapies, including but not limited to chemotherapies, surgical interventions, and radiation therapy. Chemotherapeutic agent includes all conventional cytotoxic and cytostatic agents used in cancer treatment and prevention including, from a mechanism of action standpoint; Tubulin interactive agents, DNA-interactive agents, antimetabolites and antifolates, antihormonals, antibiotics, antivirals, ODC inhibitors, and other cytotoxic agents, and prodrugs.

The nanoparticles disclosed herein could be administered in combination with other methods of radiosensitization. For example, cytokines are a class of molecules that, in some cases, also can act as radiosensitizing agents.

It will be recognized by those of skill in the art that the form and character of the particular dosing regime employed in the method of the invention will be dictated by the route of administration and other well-known variables, the sex and size of the individual, and the type and stage of the particular disease or disorder being treated. Based on such criteria, one skilled in the art can determine an amount of a composition described herein that will be effective for radiosensitization.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Targeted Gold Nanoparticle Synthesis

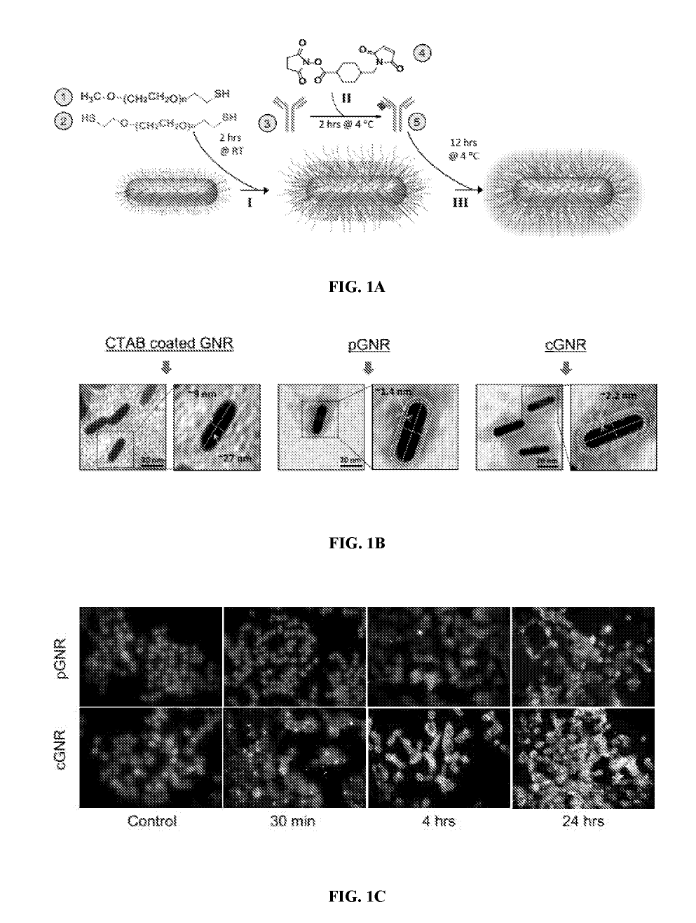

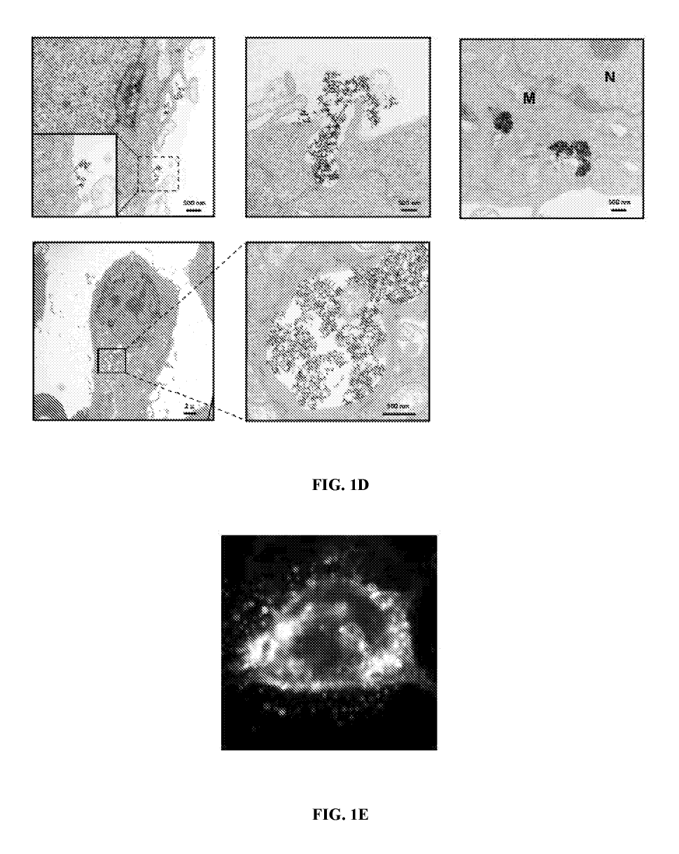

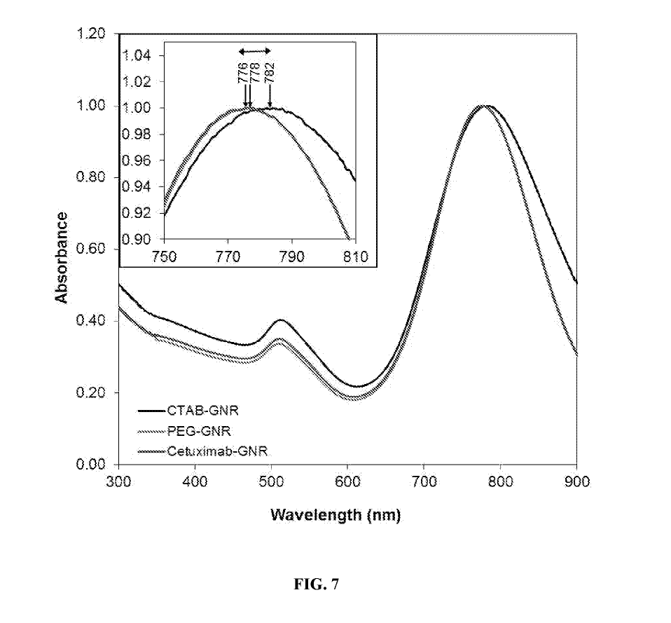

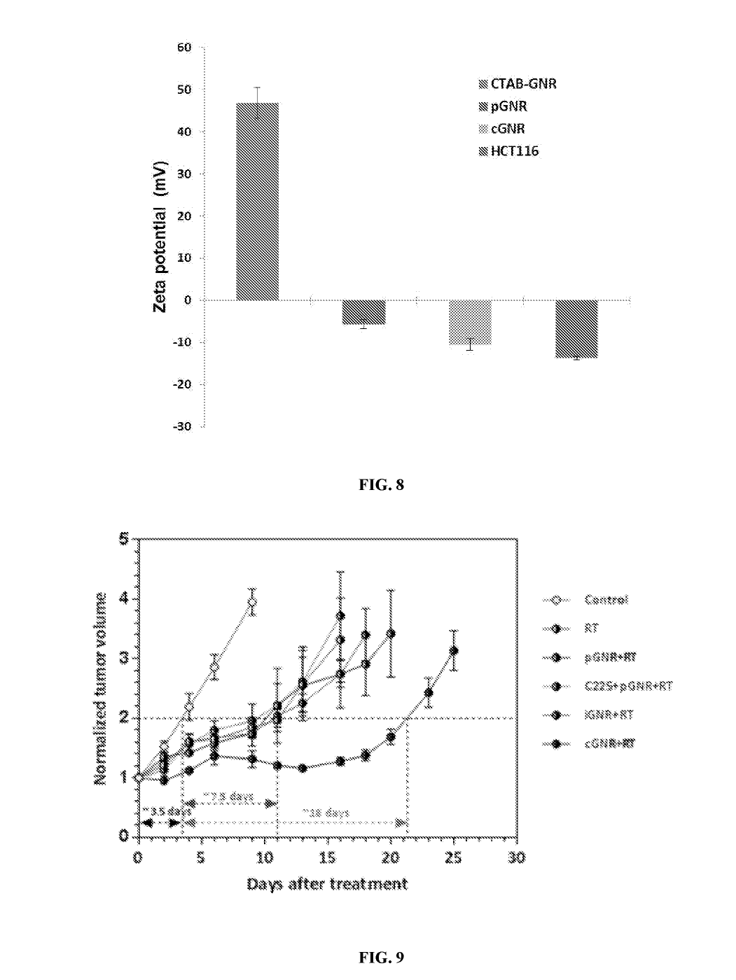

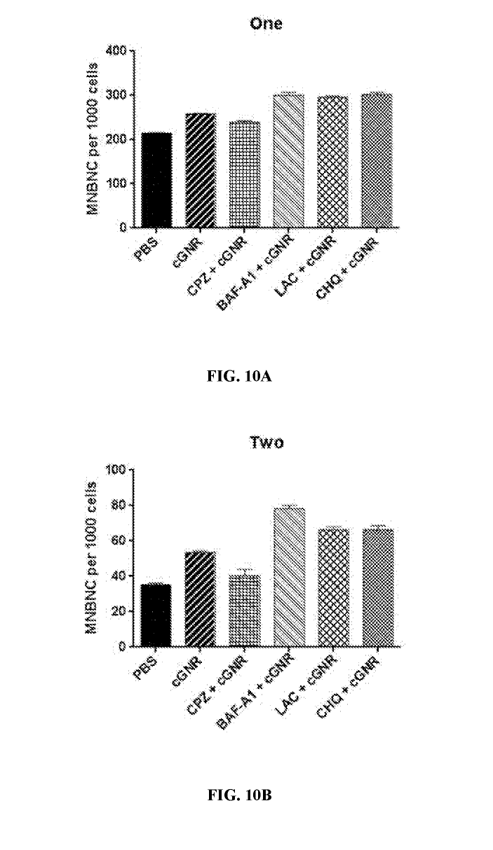

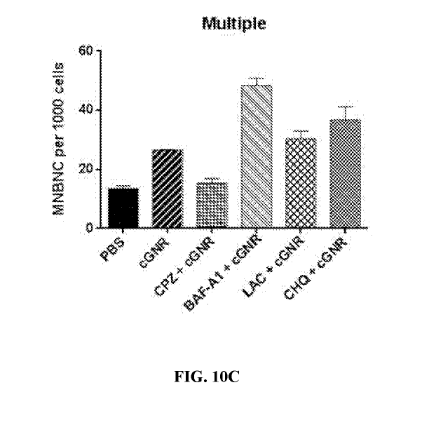

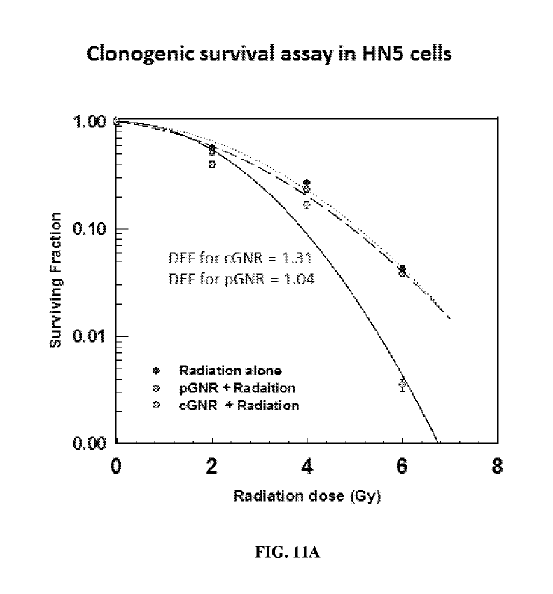

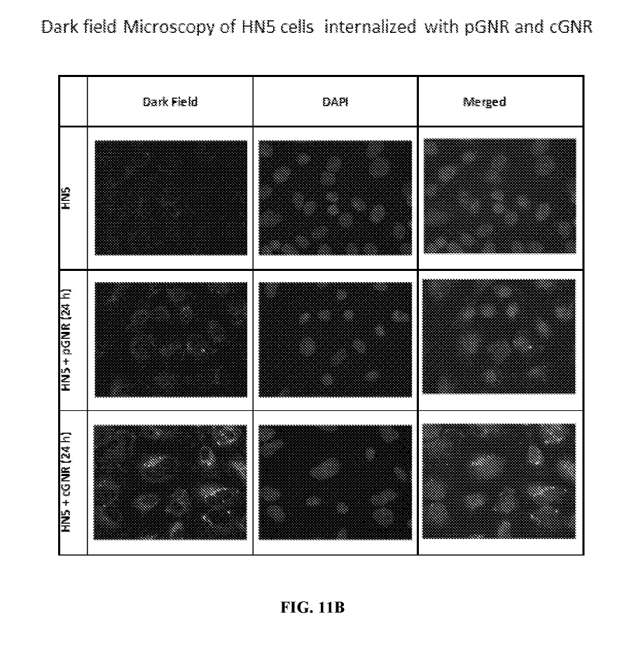

A modified seed-growth method (Zharov, et al, 2005) was used to create targeted gold nanorods (GNRs). The GNRs were synthesized with an aspect ratio of 3 and a strong longitudinal plasmon resonance around 782 nm (λmax) (FIG. 7) and stabilized with 2 mM methoxy-PEG-thiol (MW 2000) to create pegylated GNRs (pGNRs). In parallel, a 4:1 mixture of 2 mM methoxy-PEG-thiol (MW 2000) and PEG-bis-thiol (MW 5000) which yielded an average (±SE) free thiol concentration (estimated by Ellman's test) of 20.7±1.8 μM was conjugated with 11.5±1.0 μM of maleimide-activated cetuximab to create cGNRs (FIG. 1A). The highly positive zeta potential of bare GNRs (47±4 mV) due to the cationic quaternary ammonium groups of the adsorbed cetyl-trimethyl-ammonium bromide (CTAB) on their surface was neutralized by PEGylation and cetuximab conjugation (FIG. 8). Transmission electron microscope (TEM) images of negatively stained cGNRs revealed a distinct halo around the GNRs corresponding to the surface PEG and cetuximab coatings (FIG. 1B). UV-Vis absorbance measurements demonstrated a distinct spectral shift in the λmax (FIG. 7). The cGNRs demonstrated excellent stability with no apparent aggregation or change in the ζ and λmax when stored at 4° C. over a period of 24 weeks. The number of antibodies bound to each GNR was quantitatively estimated as ˜125±10 cetuximab/GNR using micro bicinchoninic acid (BCA) protein assay. The active targeting of synthesized constructs was examined in EGFR-positive HCT116 cells using dark field microscopy (DFM) and TEM. Binding and internalization of pGNRs was sporadic and non-specific, while cGNRs demonstrated rapid and stable cell surface binding and intracellular accumulation on DFM (FIG. 1C). Temporal TEM images suggested sequential receptor-mediated internalization of cGNRs culminating with substantial accumulation of cGNRs in endocytotic vesicles localized in the cytoplasmic and pen-nuclear region at 24 hrs post-incubation (FIG. 1D), corroborating the DFM findings (FIG. 1E). The cellular uptake of pGNRs and cGNRs were quantified as ˜12,000 and 31,000 per cell, respectively (FIG. 10). EGFR specificity of the cGNRs was validated by demonstrating blockade of cGNR internalization when receptors were saturated by pretreatment with cetuximab.

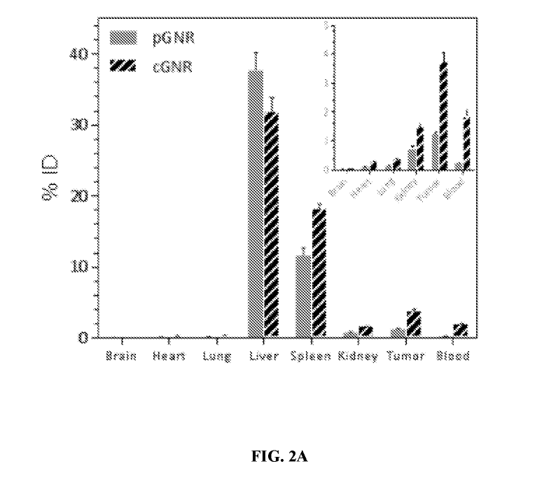

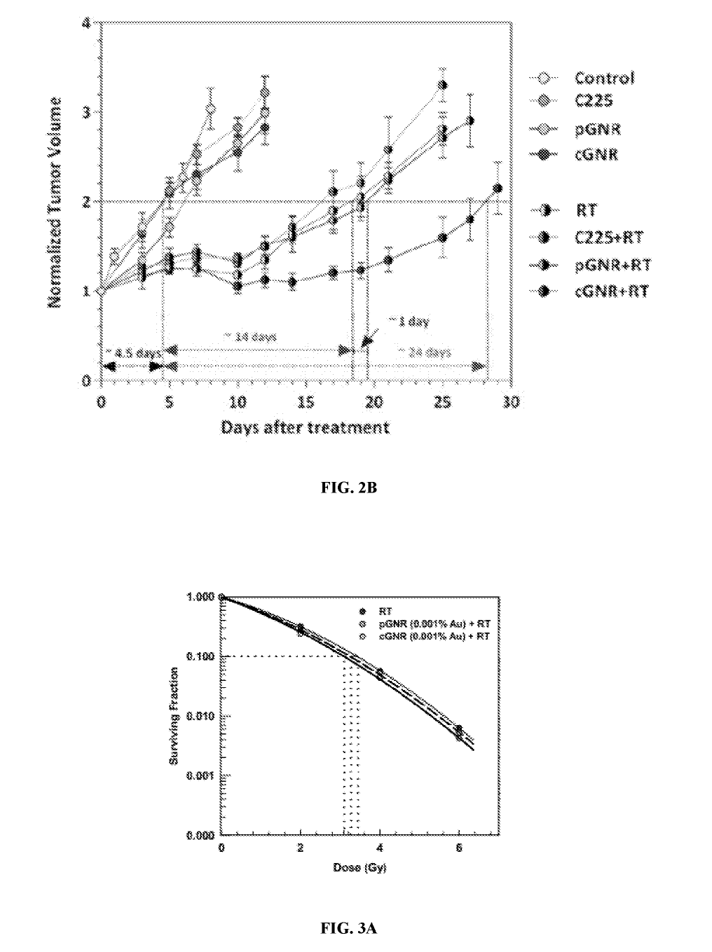

To demonstrate the translatability of the cGNR, the biodistribution and tumor accumulation was examined in HCT116 xenografts in nu/nu mice using inductively coupled plasma-mass spectrometry (ICP-MS). Liver, spleen, kidney, and tumor demonstrated significant uptake when compared to brain, heart, and lung (FIG. 2A). The enhanced uptake of pGNR and cGNRs by liver and spleen is attributed to the phagocytic clearance by the reticulo-endothelial system (RES). ICP-MS revealed significant enhancement in the tumor uptake of cGNRs (˜3.7±0.45% of injected dose (ID) vs. ˜1.2±0.08% for pGNRs). The enhanced tumor uptake is attributed to the marginalization of cGNRs along the edge of the vascular lumen for an enhanced extravasation through vascular fenestrations (Pluen, et al, 1999; Geng, et al, 2007; Lee, et al, 2009). While previous studies have demonstrated enhanced tumor uptake of rod shaped GNPs (untargeted) (Chang, et al, 2008), the concentrations required to achieve equivalent tumor uptake is ˜55-fold higher than the concentrations used in the current study.

The therapeutic efficacy of cGNR radiosensitization was assessed by tumor doubling time and animal survival. While no significant change in the tumor volume doubling time was observed with the combination of pGNR and radiation, when compared to radiation alone (250 kVp), a significant delay in the tumor volume doubling time was observed with the combination of cGNR and radiation (FIG. 2B, enhancement factor of 1.07 vs. 1.71; P<0.001). Cetuximab, either alone or in combination with radiation did not prolong the tumor growth delay when compared to the control and radiation groups, respectively. This result suggests that the presence of cetuximab in cGNRs merely mediates the tumor specific delivery of GNRs with no significant influence on the radiosensitization. Similar to lack of radiosensitization with pGNRs, no radiosensitization was observed in vivo when non-specific IgG-conjugated GNRs or a mixture of pGNRs and cetuximab was used prior to radiation therapy (FIG. 9).

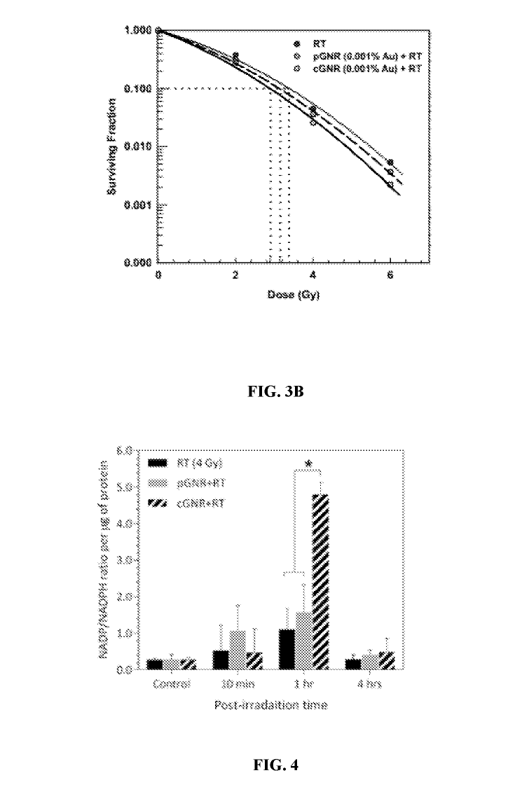

To understand the mechanism by which cGNR sensitizes tumors to radiation, in-cell assays were performed using the HCT116 cell line. In the clonogenic cell survival assay with 250 kVp x-rays, the surviving fraction of cells treated with cGNRs (0.001% wt/vol gold) for 30 min or 24 hr was substantially lower than the surviving fraction of cells that were either untreated (radiation alone) or treated with pGNR (FIGS. 3A and 3B). The estimated dose enhancement factor at 10% survival (DEF10) was 1.06 for pGNRs following both 30 min and 24 hr incubation whereas these values were 1.08 and 1.17, respectively, following 30 min and 24 hr incubation with cGNRs [p=0.00002]. This enhanced cell-kill is attributed to the localized radiation dose enhancement caused by the internalized (receptor-mediated) cGNRs that are in close proximity to the nucleus and critical cellular organelles.

### Example 2—cGNRs Enhance Radiation-Induce Cellular Oxidative Stress

Cellular redox balance, which is primarily determined by the NADP+/NADPH (nicotinamide adenine dinucleotide phosphate/reduced nicotinamide adenine dinucleotide) ratio, is crucial for cell survival. Ten minutes after a single dose of 4 Gy radiation, all treatment groups demonstrated a slight increase in their NADP+/NADPH ratio with no significant differences between the groups. However, at 1 hour post-irradiation the NADP+/NADPH ratio of the cGNR group increased 3-fold when compared to the other two groups (FIG. 4). Four hours post-irradiation, the ratio decreased to baseline levels for all treatment groups. It is inferred that the increase in cellular NADP+/NADPH ratio at 1 hour post-irradiation with cGNR is predominantly due to a transient spike in oxidative stress associated with the depletion of NADPH.

### Example 3—Internalization of cGNRs Due to End Ocytosis can be Exploited to Further Enhance the Radiosensitization

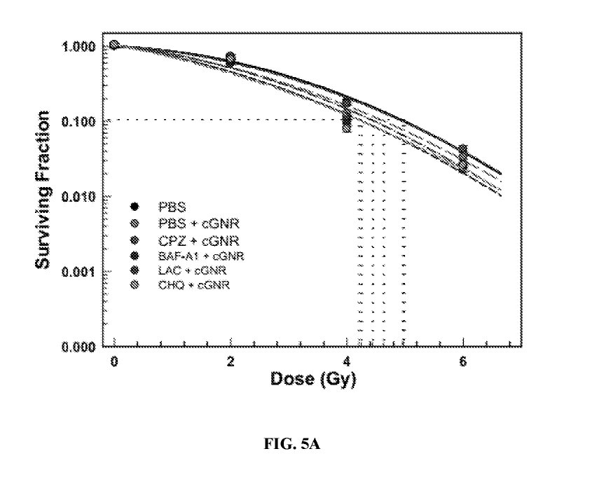

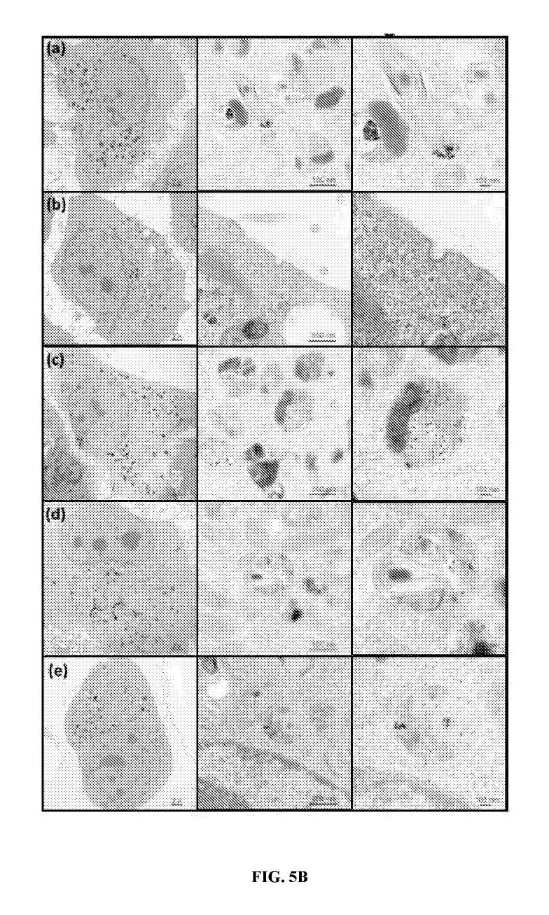

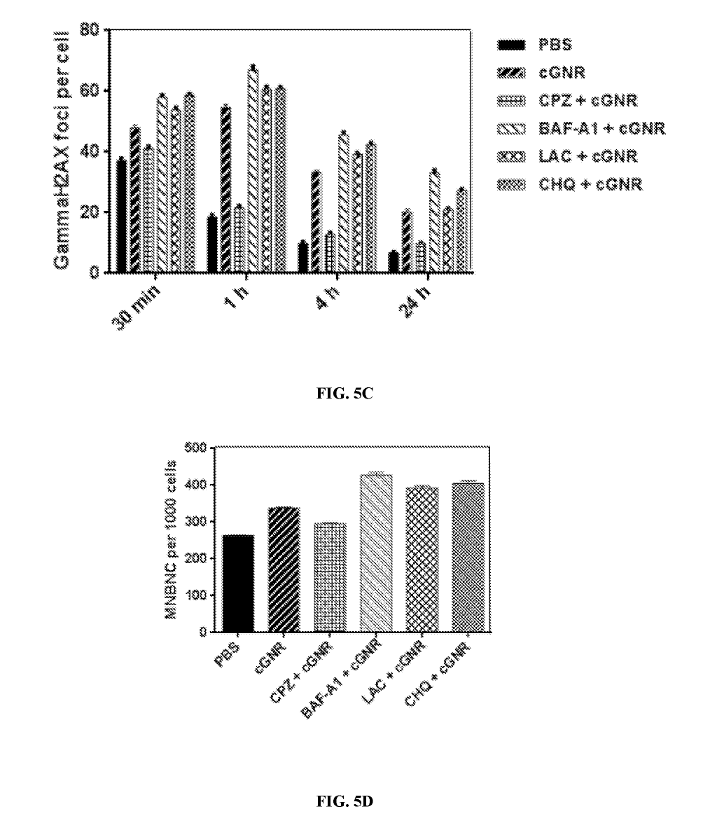

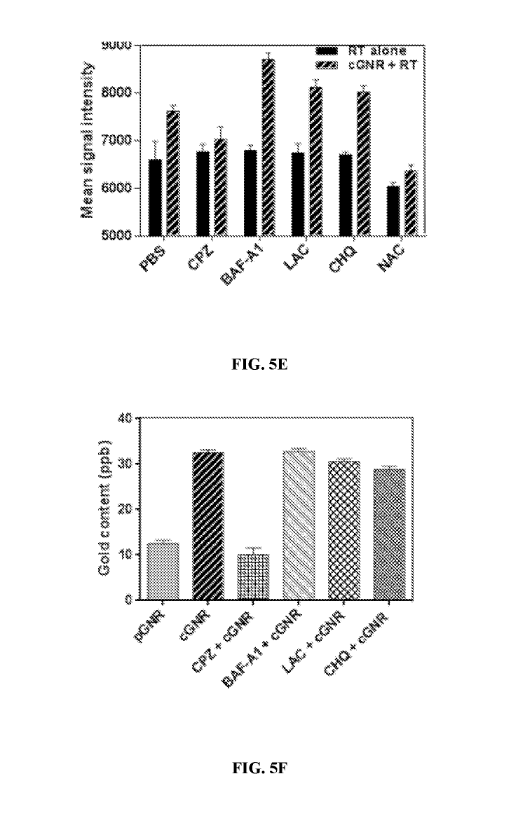

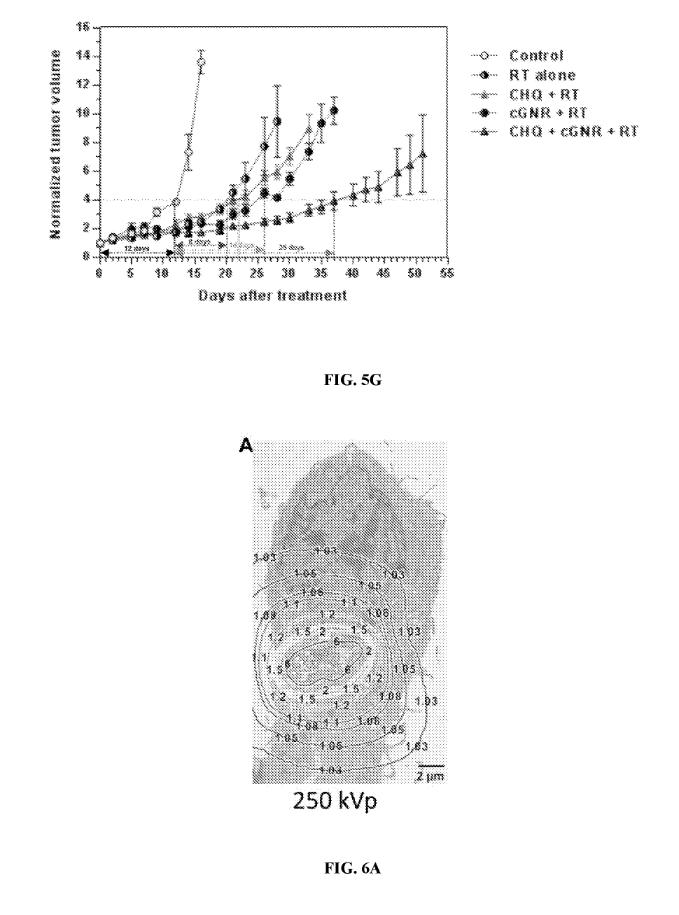

To study the effect of each step of cGNR internalization on radiosensitization, each step of the endocytotic pathway (energy-dependent receptor-mediated endocytosis, early endosomal entrapment, stasis in the late endosomal compartment, and endolysosomal fusion) was blocked using pharmacologic inhibitors in HN5 cells. The colony formation assay revealed that chlorpromazine (5 μg/ml), the inhibitor of endocytosis, did not change the cGNR-mediated dose enhancement (DRF10=1.005) (FIG. 5A). The presence of the vacuolar proton-adenosine triphosphatase (H+-ATPase) inhibitor bafilomycin A (100 nM), that lowers endosomal pH in the early endosome, enhanced the radiosensitization by cGNRs (DEF10=1.19). Since the endocytotic pathway involves transition from early endosomes to lysosomes via a proteasome or late endosome (multivesicular body), the effect of lactacystin (1 μM), a proteasome inhibitor, and chloroquine (5 μM), a lysosomotropic agent, were also evaluated. Interestingly, both agents enhanced the radiosensitization demonstrating DEF10s of 1.16 and 1.2, respectively. TEM studies suggest that vacuolar acidification results in dispersal of nanoparticles within endosomes whereas inhibition of internalization results in absence of nanoparticles within endosomes (FIG. 5B).

The results of the colony formation assay suggested that blocking steps of the endocytotic pathway would enhance the cGNR-mediated DNA damage. Therefore, γ-H2AX was evaluated in HN5 cells pretreated with different pharmacological inhibitors and incubated with cGNR for 24h followed by 2 Gy of radiation (250 kV, 15 mA) (FIG. 5C). A higher number of γ-H2AX foci per cell were observed at all-time points tested in the cGNR treated cell as compared to PBS treated and irradiated cells. Chlorpromazine pretreatment abrogated the effect of cGNR treatment by blocking the internalization of cGNR.

Bafilomycin, lactacystin, or chloroquine treatment further enhanced the DNA damage in the cGNR treated cells, with bafilomycin A demonstrating the greatest increase in DNA damage. The persistence of DNA damage was confirmed by higher number of micronuclei in the binucleated cell (MNBC) as compared to irradiated controls (FIG. 5D). The number of micronuclei in the binucleated cell shows the severity of DNA damage (one<two<multiple) (FIG. 10). Bafilomycin, lactacystin and chloroquine showed significantly higher number of MNBC as compared to irradiated control or cGNR and radiation treated cells. To further evaluate this conclusion, it was postulated that endocytosis inhibitors should demonstrate an increase in reactive oxygen species (ROS) following treatment with cGNR and radiation. Combined cGNR and radiation in HN5 cells enhanced the intracellular ROS. In agreement with our cell survival and DNA damage assay, chlorpromazine reduced the intracellular ROS, suggesting that cellular internalization of cGNR is essential for the enhancement of radiosensitization (FIG. 5E). Bafilomycin A, lactacystin, or chloroquine further enhanced the intracellular ROS over cGNR and radiation treatment. These observations suggest that disaggregation of cGNR could be further exploited for enhancement of radiosensitization.

### Example 4—Quantification of Intracellular Gold Nanorods by ICP-MS

To examine if the observed radiosensitization enhancement with bafilomycin, lactacystin, or chloroquine is due to higher uptake of GNRs, the cellular uptake of cGNR was assessed by ICP-MS. Following 24 h treatment, the increase in cellular uptake was confirmed in antibody conjugated GNRs in HN5 cells (FIG. 5F). As expected chlorpromazine significantly (p<0.001) inhibited the cellular uptake of cGNRs as compared to cGNR alone treatment group. There were no significant differences in the cellular uptake of cGNR in bafilomycin A, lactacystin, or chloroquine treated groups. These results suggest that radiation dose enhancement is not due to higher uptake but instead due to increased potency of internalized cGNRs.

### Example 5—Effect of Lysomotropic Agent on cGNR Mediated Tumor Regression

The surprising results regarding the impact of cGNR disaggregation on further radiosensitization prompted the possibility of leveraging this mechanism to develop clinically translatable therapeutic combinations. To accomplish this, the effect of lysosomotropic agent chloroquine, which is FDA approved for the treatment of malaria and rheumatoid arthritis, on cGNR mediated delay in tumor regrowth in nu/nu mice was evaluated. Similarly to HCT116 tumors (FIG. 2B), cGNR treatment significantly enhanced the radiation dose delaying the HN5 tumor regrowth by 10 days. Chloroquine concentration used in this study had a negligible effect on radiosensitization (˜2 days). Yet combining chloroquine, cGNR, and radiation demonstrated a significant delay of the tumor regrowth as compared to irradiated control (25 vs. 8 days) and as compared to mice treated with cGNR and radiation (25 vs. 14 days) (FIG. 5G).

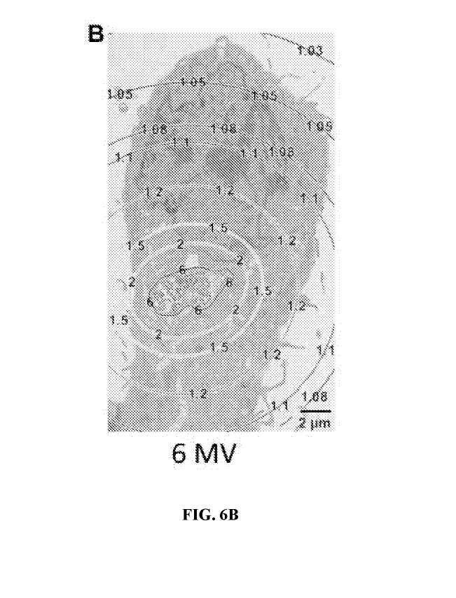

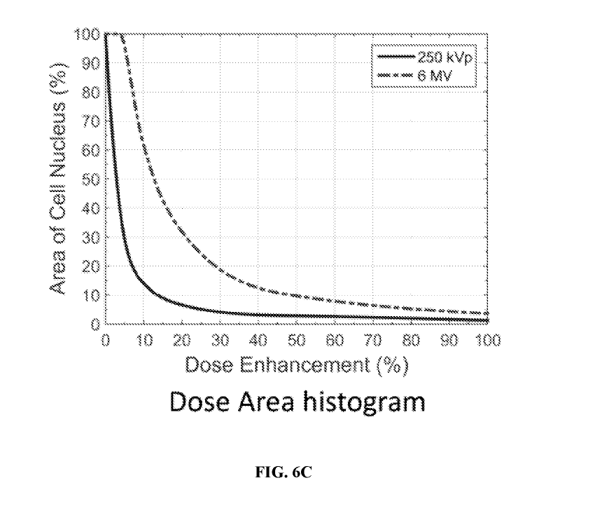

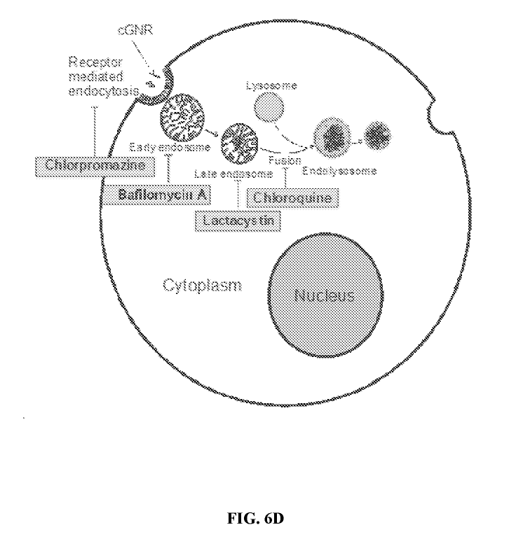

To understand the physics behind the cGNR radiosensitization, event-by-event Monte Carlo (MC) simulations were performed. A modified version of a previously reported study (Reynoso, et al, 2013), revealed a large dose enhancement (>100-fold) in the immediate vicinity (1-100 nm) of GNRs irradiated by 250 kVp x-rays (FIG. 18), while confirming a previously reported level of microscopic dose enhancement (up to 10-fold) over several microns away from GNRs for 6 MV irradiation. Attenuation of secondary electrons through the GNR clusters was taken into account by ray tracing and correction factors based on separate MC calculations quantifying the absorption of secondary electrons within finite-sized GNPs. Applying the MC-based scaled dose point kernels to a cellular geometry from a TEM image (FIG. 6A), the extent of intracellular dose enhancement due to internalized cGNRs irradiated by 250 kVp x-ray and 6 MV beams were determined (FIGS. 6A and 6B). In the presence of internalized cGNRs, both 250 kVp x-rays and 6 MV photons can induce a clinically significant level (>˜20%) of dose enhancement to some portion of the cell nucleus. The bowed shape in the dose enhancement pattern as shown for the 250 kVp x-ray source is attributed to more pronounced attenuation of secondary electrons by clustered GNRs, compared to the 6 MV source. More quantitative observations about the physical dose enhancement to the nucleus can be made from the dose enhancement-area histogram plot (FIG. 6C). In general, while the overall fluence of secondary electrons from GNRs is much larger for 250 kVp x-rays, the range of such secondary electrons are much longer for 6 MV photons. Thus, most of the secondary electrons from 250 kVp irradiation lose their energy in the vicinity of GNRs (or clustered GNRs), when compared to those generated from 6 MV irradiation. Accordingly, despite their ability to produce significantly more low energy secondary electrons, 250 kVp x-rays become less efficient than 6 MV photons in terms of inducing physical dose enhancement at sites away from the locations of GNRs. Nonetheless, clinically significant microscopic dose enhancement observed over ˜3 μm from the GNR clusters demonstrates that the radiosensitization effect is dependent on the microscopic localization and/or distribution of cGNRs in and around the tumor cell. Overall, the current results suggest that the secondary electrons generated in the immediate vicinity of cGNRs are absorbed within the short range by interacting with critical cellular organelles, thereby initiating a cascade of biological events resulting in an enhanced cell death caused in synergy with the nuclear DNA damage.

Use of nanoparticles in the clinic has been lagging due to failure to demonstrate translatable preclinical impact. Radiation is a mainstay therapy for the majority of solid tumors but dose limitations remain due to toxicity to surrounding healthy tissue. In this work, the potential clinical use of newly developed gold nanoconstructs for radiation dose escalation in solid tumors has been demonstrated. The cetuximab-conjugated nanorods (cGNR) induced more DNA damage and increased levels of reactive oxygen species that resulted in increased cell death and significantly slower tumor growth. The mechanism of action was dependent on internalization and the effect was dramatically improved by dispersion of particles in the cytoplasm. This discovery has led to the proposal of a brand new combination therapy using an antimalarial drug with targeted gold nanorods and radiation that could be effective for treatment of solid tumors.

### Example 6—Synthesis and Conjugation Methods

Gold Nanorod Synthesis: Gold nanorods of approximately 27 nm×9 nm in size were synthesized using a seeded growth method. Gold nanorod seed was prepared by stirring 100 mM CTAB and 10 mM chloro-auric acid, and reducing with 100 mM sodium borohydride solution. The seed was allowed to age for 2 hours. The growth solution was then prepared by allowing a mixture of 100 mM CTAB and 10 mM chloro-auric acid react with 10 mM silver nitrate, 100 mM ascorbic acid and the prepared nanorod seed solution while stirring rapidly for 2 minutes and leaving the solution to sit for 15 minutes at 25° C. The growth solution was eventually chilled to 4° C. and run through a tangential flow filter to extract excess CTAB crystals.

Preparation of Nanorods for Conjugation:

The level of CTAB present in the solution was assessed via a test for detection of cationic active matter. The nanorods were diluted to bring the CTAB level to 1 mM. The rods were initially conjugated with 2 mM hydrazide-PEG-SH, adding 10 μL of PEG for every milliliter of nanorod solution. The solution was allowed to incubate at room temperature overnight and washed in 1×PBS the following day to eliminate any unbound PEG. Further, 5 mM mPEG-SH was added as a blocking agent at 4 μL/mL and allowed to react for 30 minutes. The rods were washed in 1×PBS to get rid of unbound PEG.

Antibody Activation & Conjugation to Nanorods:

100 μL of Cetuximab (2 mg/mL) was diluted in 1×PBS to bring its concentration down to 0.1 mg/mL, and activated following the addition of 20 μL of 100 mM sodium periodate and incubation in a dark environment for 30 minutes. 100 μL of the activated antibody was later added to every 1 mL of PEGylated gold nanorod particles and allowed to stir for 2 hours at 4° C. Conjugated nanorods were centrifuged at 4° C. to remove any excess unbound antibody and concentrated as desired.

Gold Colloid Synthesis:

Gold colloid, approximately 30 nm in size, was synthesized by a seeded growth method using smaller gold colloid particles as a precursor. 12 nm gold colloid was prepared by the rapid reduction of 100 mL of 1 mM chloro-auric acid by 10 mL of 38.8 mM sodium citrate solution (Preparation and Characterization of Au Colloid Monolayers, Graber, K. C. et al (1995), incorporated herein by reference). Further, 2 mL of the prepared 12 nm gold colloid was diluted to 100 mL and brought to a boil with vigorous stirring. Upon boiling, 1 mL of 1% chloro-auric acid and 0.5 mL of 38.8 mM sodium citrate solution were added simultaneously yet rapidly, and stirred over heat until the color stabilized to burgundy.

Preparation of Gold Colloid for Conjugation:

The particles were initially conjugated with 2 mM hydrazide-PEG-SH, adding 10 μL of PEG for every milliliter of gold colloid solution at 1 OD. The solution was allowed to incubate at room temperature overnight and washed in 1×PBS the following day to eliminate any unbound PEG. Further, 5 mM mPEG-SH was added as a blocking agent at 4 μL/mL/OD and allowed to react for 30 minutes. The particles were washed in 1×PBS to get rid of unbound PEG.

Antibody Activation & Conjugation to Gold Colloid:

1004, of Cetuximab (2 mg/mL) was diluted in 1×PBS to bring its concentration down to 0.1 mg/mL, and activated following the addition of 20 μL of 100 mM sodium periodate and incubation in a dark environment for 30 minutes. 100 μL of the activated antibody was later added to every 1 mL of PEGylated gold colloid particles and allowed to stir for 2 hours at 4° C. Conjugated nanoparticles were centrifuged at 4° C. to remove any excess unbound antibody and concentrated as desired.

### Example 7—Additional Synthesis and Conjugation Methods

In another example, the antibody was directionally conjugated to the nanorod to allow a more efficient presentation for cellular targeting,

Gold Nanorod Synthesis:

Gold nanorods of approximately 26 nm×8 nm in size were synthesized using a seeded growth method. Gold nanorod seed was prepared by stirring 100 mM CTAB and 10 mM chloro-auric acid, and reducing with 100 mM sodium borohydride solution. The seed was allowed to age for 2 hours. The growth solution was then prepared by allowing a mixture of 100 mM CTAB and 10 mM chloro-auric acid react with 10 mM silver nitrate, 100 mM ascorbic acid and the prepared nanorod seed solution while stirring rapidly for 2 minutes and leaving the solution to sit for 15 minutes at 25° C. The growth solution was eventually chilled to 4° C. and run through a tangential flow filter to extract excess CTAB crystals.

Preparation of Nanorods for Conjugation:

The level of CTAB present in the solution was assessed via a test for detection of cationic active matter. The nanorods were diluted to bring the CTAB level to 1 mM. The rods were initially conjugated with 2 mM hydrazide-PEG-SH, adding 10 μL of PEG for every milliliter of nanorod solution. The solution was allowed to incubate at room temperature overnight and washed in 1×PBS the following day to eliminate any unbound PEG. Further, 5 mM mPEG-SH was added as a blocking agent at 4 μL/mL and allowed to react for 30 minutes. The rods were washed in 1×PBS to get rid of unbound PEG.

Antibody Activation & Conjugation to Nanorods:

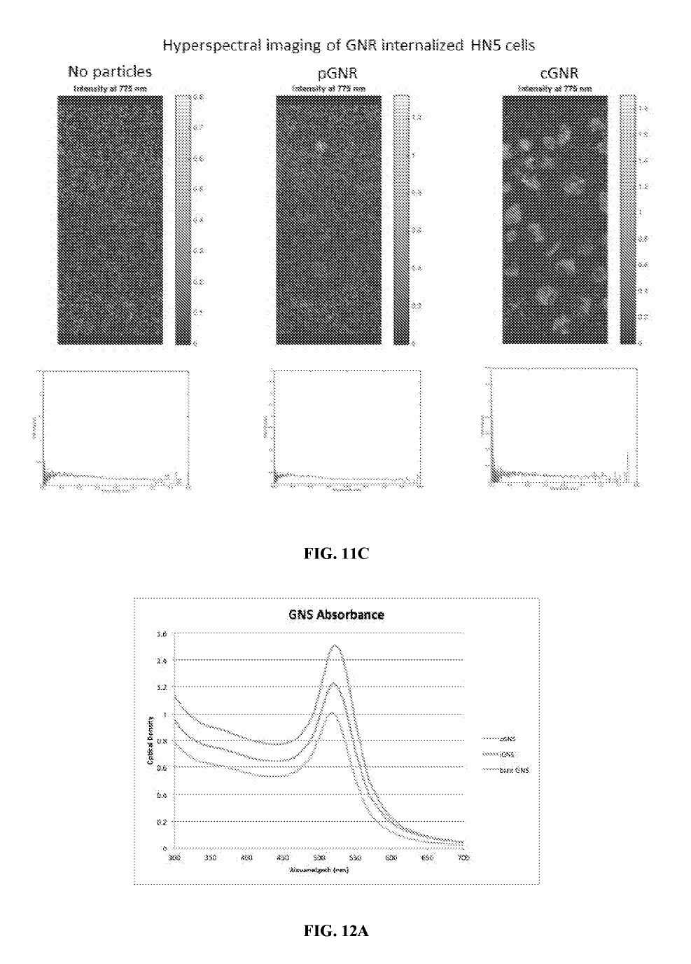

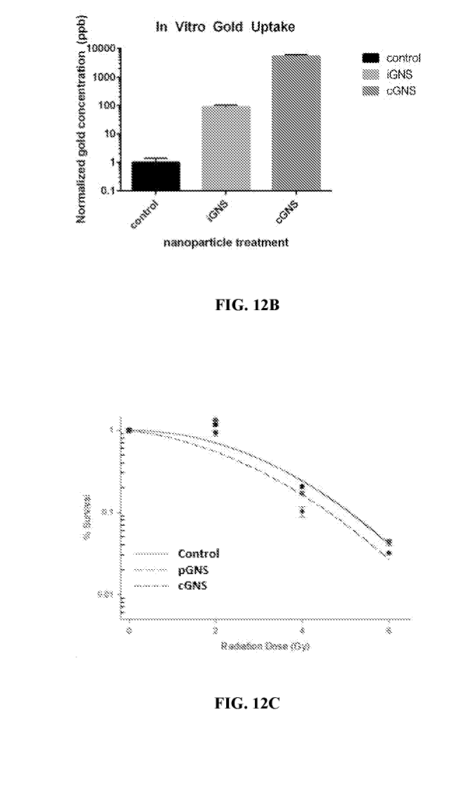

100 μL of Cetuximab (2 mg/mL) was diluted in 1×PBS to bring its concentration down to 0.1 mg/mL, and activated following the addition of 20 μL of 100 mM sodium periodate and incubation in a dark environment for 30 minutes. 100 μL of the activated antibody was later added to every 1 mL of PEGylated gold nanorod particles and allowed to stir for 2 hours at 4° C. Conjugated nanorods were centrifuged at 4° C. to remove any excess unbound antibody and concentrated as desired. The radiation dose enhancement using the directionally conjugated antibody is illustrated in FIG. 11.

### Example 8—Radiosensitization Using Spherical Gold Nanoparticles

As described herein, the enhancement of radiation is dependent on the presence of high-Z materials. As another example, 30 nm gold colloid or gold nanospheres (GNS) conjugated with an anti-EGFR antibody was used to enhance the effects of radiation. 30 nm citrate-coated gold nanospheres were obtained from NanoHybrids. Cetuximab-conjugated GNS (cGNS) were produced by directionally conjugating cetuximab via a hydrazine linker. Cetuximab suspended in 100 uL of 100 mM Na2HPO4, pH7.5 was initially reacted with 10 uL of 100 mM NaIO₄ in the dark for 30 min at RT and quenched with 500 uL of 1×PBS before being incubated with 2 uL of 46.5 mM dithiolaromatic PEG6-CONHNH₂ in deionized, ultrafiltered water for 1 h at RT. 1 ml of 40 mM HEPES was added to this and filtered in a 10 k MWCO centrifuge filter (Millipore) at 2,000 g at 4° C. until ˜75% of the solution has passed through the filter (about 10 min). The retained solution was resuspended in 40 mM HEPES to a final volume of 1 ml and an antibody concentration of 100 μg ml−1. 100 μl of antibody-linker solution (100 μg ml−1) was incubated with 1 ml of 30 nm colloidal gold solution for 20 min at RT on a rotator.

Non-specific whole human IgG-conjugated gold nanospheres serving as control (iGNS) were generated by conjugated thiol-PEG-COOH with the antibody using EDC-NHS chemistry. Briefly, bare nanospheres were combined with 1 mM thiol-PEG-COOH at a ratio of 15 ul per OD*ml of nanospheres and reacted at room temperature on a rotator for 4 hours. The pegylated nanospheres were then centrifuged at 6500 g for 30 minutes and resuspended in milli Q water to 50 OD. To activate the COOH group, 10 ul of 50 OD pegylated nanospheres were combined with EDC/sulfo-NHS in MES buffer (30 mg/ml, pH 6.8) in a 1:1 ratio and incubated for 30 m at room temperature. After activation, 1 ml of PBS was added to the reaction and the entire volume was centrifuged. After discarding the supernatant, 10 ul of human IgG (1 mg/ml in PBS) was added to the activated pegylated nanospheres and allowed to react at room temperature for 2 hours. To remove excess reagents, the final iGNS conjugates were concentrated by centrifugation, resuspended in PBS, and centrifuged again. The properties of the produced GNSs were then evaluated by spectrometry and are shown below in Table 1 and FIG. 12A. Furthermore, a Micro-BCA assay estimate of number of cetuximabs per GNS as 327.

TABLE 1
GNS properties

| Characteristic/Particle | Bare GNS | | iGNS | | cGNS | |
|---|---|---|---|---|---|---|
| DLS size | 26.73 | nm | 53.79 | nm | 70.92 | nm |
| Zeta potential | −53.73 | mV | −5.662 | mV | −5.38 | mV |

In vitro gold uptake was measured in HN5 head and neck cancer cells by inductively coupled plasma mass spectrometry after treating cells in culture with 1 OD nanoparticle solutions for 24 h and normalizing for cell number. Results of these studies are shown in FIG. 12B. To visualize binding of cGNS and iGNS to HN5 cells, dark field imaging was also performed on cells grown in chamber slides. These studies demonstrated potent binding of cGNS but not iGNS to EGFR-expressing cells.

cGNS and pGNS (pegylated GNS) were further evaluated for their ability to sensitize HN5 cells to radiation in clonogenic assays where cultured cells were exposed varying doses of radiation 24 h after treatment with 0.5 OD nanoparticles. Colonies were counted 14 days following radiation and normalized for plating efficiency. Briefly, HN5 cells were seeded in petri dishes for 48 h. Later, at 70% confluence cells were incubated with PBS, pGNR (OD=0.5), or cGNR (OD=0.5) for 24 h. Cells were irradiated with 2, 4, and 6 Gy of X-Ray (250 kVP, 15 mA). After irradiation known number of HN5 cells were seeded in six well plates and incubated for 14 days to count colonies. As shown in FIG. 12C, only conjugated GNSs but not pegylated GNSs potently sensitized cells to radiation.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Dvorak, H. F. et al., Am J Pathol 133(1): 95-109, 1988.
Maeda, H. et al., Int Immunopharmacol 3(3): 319-28, 2003.
Huang, X. et al., Nanomedicine (Lond) 2(5): 681-93, 2007.
Caliceti, P. and Veronese, F. M., Adv Drug Deliv Rev 55(10): 1261-77, 2003.
Mitragotri, S. and Lahann, J., Nat Mater 8(1): 15-23, 2009.
Lee, S. Y. and Ferrari, M. et al., Nanotechnology 20(49): 495101, 2009.
Bonner, J. A. et al., N Engl J Med 354(6): 567-78, 2006.
Goldberg, R. M., Nat Rev Drug Discov Suppl: S I 0-1, 2005.
Graber, K. C. et al. Preparation and Characterization of Au Colloid Monolayers, Anal. Chem., 67(4):735-743, 1995.
Huff, T. B. et al., Langmuir 23(4): 1 596-9, 2007.
Hauck, T. S. et al., Small 4(1); 153-9, 2008.
Unezaki, S. et al., Int. J. Pharm 144: 11-17, 1996.
Hainfeld J F, Slatkin D N, Smilowitz H M. The use of gold nanoparticles to enhance radiotherapy in mice. Phys Med Biol 2004; 49:N309-15.
Hainfeld J F, Dilmanian F A, Slatkin D N, Smilowitz H M. Radiotherapy enhancement with gold nanoparticles. J Pharm Pharmacol 2008; 60:977-85.
Joh D Y, Sun L, Stangl M, Al Zaki A, Murry S, Santoiemma P P, Davis J J, Baumann B C, Alonso-Basanta M, Bhang D, Kao G D, Tsourkas A, Dorsey J F. Selective targeting of brain tumors with gold nanoparticle-induced radiosensitization. PLoS One. 2013 Apr. 30; 8(4):e62425.
Zharov V P, Kim J W, Curiel D T, Everts M. Self-assembling nanoclusters in living systems: application for integrated photothermal nanodiagnostics and nanotherapy. Nanomedicine 2005; 1:326-45.
Geng Y, Dalhaimer P, Cai S, et al. Shape effects of filaments versus spherical particles in flow and drug delivery. Nat Nanotechnol 2007; 2:249-55.
Pluen A, Netti P A, Jain R K, Berk D A. Diffusion of macromolecules in agarose gels: comparison of linear and globular configurations. Biophys J 1999; 77:542-52.
Lee S Y, Ferrari M, Decuzzi P Shaping nano-/micro-particles for enhanced vascular interaction in laminar flows. Nanotechnology 2009; 20:495101.
Chang M Y, Shiau A L, Chen Y H, Chang C J, Chen H H, Wu C L. Increased apoptotic potential and dose-enhancing effect of gold nanoparticles in combination with single-dose clinical electron beams on tumor-bearing mice. Cancer Sci 2008; 99:1479-84.
Reynoso, F., Manohar N., Krishnan S., and Cho, S. H. Design of a Yb¹⁶⁹ Source Optimized for Gold Nanoparticle-Aided Radiation Therapy, 41(10):101709, Medical Physics, 2014

What is claimed is:

1. A method sensitizing target cells in a subject to a radiation therapy comprising:
administering to the subject a effective amount of high-Z particles, said high-Z particles comprising a targeting molecule that binds to target cells and a high-Z element; and
administering to the subject an effective amount of a de-aggregation agent that reduces intracellular aggregation of said high-Z particles.

2. The method of claim 1, wherein the de-aggregation agent is selected from the groups consisting of a lysosomotropic agent, an endosome acidification inhibitor, a vacuolar proton-adenosine triphosphate inhibitor, a proteasome inhibitor, a cathepsin A inhibitor, an intralysosomal proteolysis inhibitor, an intracellular vesicular swelling promoter and an inhibitor of late endosome lysosome fusion.

3. The method of claim 1, further comprising irradiating the target cells with ionizing radiation.

4. The method of claim 3, wherein ionizing radiation is delivered as a plurality of doses over a period of time.

5. The method of claim 1, wherein the targeting molecule mediates internalization of the high-Z particles upon binding with the targeted cells.

6. The method of claim 1, wherein the high-Z particles are administered to the subject two or more times.

7. The method of claim 1, wherein the target cells are irradiated within the subject.

8. The method of claim 1, wherein the target cells are circulating cells.

9. The method of claim 1, wherein the target cells are endothelial cells or stromal cells.

10. The method of claim 1, wherein the target cells comprises cancer cells.

11. The method of claim 10, wherein the cancer cells are primary cancer cells.

12. The method of claim 10, wherein the cancer cells are metastatic cancer cells.

13. The method of claim 10, wherein the cancer cells are selected from the group consisting of brain, lung, liver, spleen, kidney, lymph node, small intestine, pancreas, blood, colon, stomach, breast, endometrium, prostate, testicle, ovary, skin, head and neck, esophagus, lymphatic, bone marrow and bone cancer cells.

14. The method of claim 1, wherein the target cells are irradiated extracorporeally.

15. The method of claim 1, wherein the high-Z element is gold, silver, iodine, gallium, barium, iron, gadolinium, platinum, hafnium or combinations thereof.

16. The method of claim 1, wherein the high-Z particles comprises nanorods, nanoshells, colloids, nanocages, or nanoprisms.

17. The method of claim 1, wherein the high-Z particles have an average size of between about 1 nm and 200 nm.

18. The method of claim 1, wherein the targeting molecule has an affinity for a receptor expressed in cancer cells.

19. The method of claim 1, wherein the targeting molecule binds to human epidermal growth factor receptor, human epidermal growth factor receptor 2, human epidermal growth factor receptor 3, human epidermal growth factor receptor 4, vascular endothelial growth factor receptor, folic acid receptor, melanocyte stimulating hormone receptor, integrin avb3, integrin avb5, transferrin receptor, interleukin receptors, lectins, insulin-like growth factor receptor, hepatocyte growth factor receptor or basic fibroblast growth factor receptor.

20. The method of claim 1, wherein the targeting molecule comprises an antibody, a polypeptide, a dendrimer, an aptamer, an oligomer or a small molecule.

21. The method of claim 1, wherein the targeting molecule is selected from the group consisting of: cetuximab, an EGFr-binding polypeptide, trastuzumab, folic acid, melanocyte stimulating hormone, a transferrin receptor targeted polypeptide, a transferrin receptor targeted antibody and cyclic-RGD.

22. The method of claim 3, wherein the ionizing radiation is delivered continuously through an implant of a radiation-emitting substance near the target cells.

23. The method of claim 3, wherein the irradiation is a heavy ion therapy.

24. The method of claim 23, wherein the irradiation is a proton therapy.

25. The method of claim 2, wherein the endosome acidification inhibitor comprises tamoxifen.

26. The method of claim 2, wherein the inhibitor of vacuolar-type proton-adenosine triphosphate is selected from the group consisting of bafilomycin A, concanamycin A, concanamycin B and bafilomycin B1.

27. The method of claim 2, wherein the proteasome inhibitor and cathepsin inhibitor are selected from the group consisting of lactacystin, chymostatin, clasto-Lactacystin β-lactone and MG-132.

28. The method of claim 2, wherein the inhibitor of late endosome lysosome fusion is selected from the group consisting of phosphatidylinositol 3-phosphate kinase inhibitors, Rab-GDP-dissociation inhibitor (Rab-GDI), chloroquine, and primaquine.

29. The method of claim 2, wherein the lysosomotropic agent is selected from the group consisting of acetaminophen, diclofenac, rosuvastatin, amiodarone, chloroquine, amantadine, ammonium chloride, monensin and nigericin.

30. The method of claim 1, wherein the de-aggregation agent is not acetaminophen.

31. The method of claim 1, wherein the de-aggregation agent is not chloroquine.

32. The method of claim 2, wherein the intralysosomal proteolysis inhibitor is selected from the group consisting of suramin, phosphoramidon, leupeptin, E64, pepstatin, a cystatin or a bestatin.

33. The method of claim 2, wherein the intracellular vesicular swelling inducer is selected from the group consisting of polyvinylpyrrolidone and dextran.

34. The method of claim 1, wherein the high-Z particles are administered before or essentially simultaneously with the de-aggregation agent.

35. The method of claim 34, wherein the high-Z particles and the de-aggregation agent are comprised in the same composition.

36. The method of claim 35, wherein the high-Z particles comprise the de-aggregation agent.

37. The method of claim 1, wherein the high-Z particles are administered after the de-aggregation agent.

38. The method of claim 1, further comprising imaging the high-Z particles and the target cells.

39. The method of claim 1, further comprising administering a further anti-cancer therapy to the subject.

40. The method of claim 39, wherein the further anti-cancer therapy is a chemotherapy.

41. The method of claim 1, wherein the high-Z particle further comprises a chemotherapeutic agent.

42. A method sensitizing tumor cells in a subject to a radiation therapy comprising:
administering to the subject a effective amount of high-Z particles; and
administering to the subject an effective amount of a de-aggregation agent that reduces intracellular aggregation of said high-Z particles.

43. A method sensitizing target cells in a subject to a radiation therapy comprising:
administering to the subject a effective amount of high-Z particles, said high-Z particles comprising a targeting molecule that binds to target cells and a high-Z element;
administering to the subject an effective amount of a de-aggregation agent that reduces intracellular aggregation of said high-Z particles; and
irradiating the target cells with ionizing radiation.

44. A pharmaceutical composition comprising (i) high-Z particles, said high-Z particles comprising a targeting molecule that can bind to cells and a high-Z element; and (ii) a de-aggregation agent.

45. A composition for use in sensitizing target cells in a subject to a radiation therapy, the composition comprising (i) high-Z particles, said high-Z particles comprising a targeting molecule that binds to the target cells and a high-Z element; and (ii) a de-aggregation agent.

46. A composition for use in treating a subject who has previously been administered high-Z particles, the composition comprising a de-aggregation agent.

47. A composition for use in treating a subject who has previously been administered de-aggregation agent, the composition comprising an effective amount of high-Z particles, comprising a targeting molecule that can bind to cells and a high-Z element.
